(19) United States
(12) Patent Application Publication
(10) Pub. No.: US 2024/0075296 A1
(43) Pub. Date: Mar. 7, 2024

(54) ENDOVASCULAR DEEP BRAIN STIMULATION

(71) Applicant: Covidien LP, Mansfield, MA (US)

(72) Inventors: Varun Umesh Kashyap, Irvine, CA (US); Mark P. Ashby, Laguna Niguel, CA (US); Scott R. Stanslaski, Shoreview, MN (US); Kevin V. Nguyen, Westminster, CA (US)

(21) Appl. No.: 18/454,395

(22) Filed: Aug. 23, 2023

Related U.S. Application Data
(60) Provisional application No. 63/373,606, filed on Aug. 26, 2022.
(60) Provisional application No. 63/373,612, filed on Aug. 26, 2022.
(60) Provisional application No. 63/484,049, filed on Feb. 9, 2023.

(51) Int. Cl. A61N 1/36; A61N 1/05

(52) U.S. Cl. CPC A61N 1/3614; A61N 1/0534; A61N 1/36139; A61N 1/36146

(57) ABSTRACT

In some examples, an endovascular device includes an elongated body configured to be introduced in a blood vessel of a patient and a plurality of electrodes disposed along the elongated body. The plurality of electrodes includes a first group of electrodes and a second group of electrodes. The endovascular device further includes a plurality of conductors including a first conductor electrically coupled to each electrode of the first group of electrodes and a second conductor electrically coupled to each electrode of the second group of electrodes. Each electrode of the first group of electrodes faces a first direction and each electrode of the second group of electrodes faces a second direction different from the first direction. The plurality of electrodes is configured to deliver electrical stimulation to tissue of a brain of the patient or sense a patient parameter from a location within the blood vessel.

This application claims the benefit of U.S. Provisional Application No. 63/373,612, filed Aug. 26, 2022, and entitled, “ENDOVASCULAR DEEP BRAIN STIMULATION,” the benefit of U.S. Provisional Application No. 63/373,606, filed Aug. 26, 2022, and entitled, “ENDOVASCULAR DEEP BRAIN STIMULATION,” and the benefit U.S. Provisional Application No. 63/484,049, filed Feb. 9, 2023, and entitled, “ENDOVASCULAR DEEP BRAIN STIMULATION,” the entire contents of each of which is incorporated herein by reference.

### TECHNICAL FIELD

This disclosure relates to electrical stimulation therapy.

### BACKGROUND

Medical devices, such as electrical stimulation devices, may be used in different therapeutic applications, such as deep brain stimulation (DBS). A medical device may be used to deliver therapy to a patient to treat a variety of symptoms or patient conditions such as, but not limited to, movement disorders, seizure disorders (e.g., epilepsy), or mood disorders. In some therapy systems, an implantable electrical stimulator delivers electrical stimulation therapy to a target tissue site within a patient with the aid of one or more electrodes.

### SUMMARY

This disclosure describes example endovascular devices and systems configured to deliver electrical stimulation therapy to a brain of a patient and/or sense one or more patient parameters (e.g., brain signals), as well as methods of delivering endovascular deep brain stimulation (DBS) or sensing using the devices and systems described herein. The medical devices and systems described herein are configured to be relatively minimally invasive at least because they are configured to be navigated through vasculature of a patient to an intracranial blood vessel (e.g., a venous vessel or an arterial vessel) in order to deliver electrical stimulation therapy to a target tissue site in a brain of a patient and/or sense one or more patient parameters.

In examples described herein, an endovascular device includes a plurality of segmented or partial ring electrodes and the segmented or partial ring electrodes that face in a common direction (e.g., a radial direction relative to a longitudinal axis of the endovascular device) are electrically connected together via a common electrical conductor. Electrically connecting multiple electrodes together may help reduce an overall profile of the medical device by reducing a total number of electrical conductors, e.g., as compared to medical devices in which each electrode is electrically connected to a separate electrical conductor. The electrodes that are electrically connected together are configured to be activated (e.g., to sense or deliver electrical stimulation) as a group. The medical device can include a plurality of groups of segmented or partial ring electrodes, each group including electrodes that face in a common direction and are electrically coupled together. The electrodes of one group of electrodes is electrically isolated from electrodes of another group of electrodes and each group of electrodes is independently activatable. Thus, a medical device can selectively deliver electrical stimulation or sense via one or more groups of electrodes while not delivering electrical stimulation or sensing via one or more other groups of electrodes.

The direction in which the electrodes face impacts the direction in which the electrodes deliver electrical stimulation and/or sense bioelectrical signals in a patient. Electrically connecting the electrodes that face in a common direction and enabling each group to be independently activated may provide a uniform or directional electrical stimulation field and/or facilitate directional sensing. The directional electrical stimulation field can help target certain regions of brain while minimizing delivery of electrical stimulation to other regions of brain associated with adverse effects.

In some examples, an endovascular device includes an elongated body configured to be introduced in a blood vessel of a patient, a plurality of electrodes disposed along the elongated body, and a plurality of conductors electrically coupled to different groups of electrodes of the plurality of electrodes. The plurality of electrodes is configured to deliver electrical stimulation to tissue of a brain of the patient or sense a patient parameter from a location within the blood vessel. The plurality of electrodes includes a first group of electrodes and a second group of electrodes wherein each electrode of the first group of electrodes faces a first direction and each electrode of the second group of electrodes faces a second direction different from the first direction. The plurality of conductors includes a first conductor electrically coupled to each electrode of the first group of electrodes and a second conductor electrically coupled to each electrode of the second group of electrodes. Each electrode of the plurality of electrodes is a segmented electrode or a partial ring electrode.

In some examples, an endovascular device includes an elongated body, a plurality of electrodes disposed along the elongated body, and a plurality of conductors. The elongated body includes a distal portion that is transformable between a relatively low-profile delivery configuration and a deployed (e.g., coiled or expanded) configuration and is configured to be introduced in a blood vessel of a patient. The plurality of electrodes is configured to deliver electrical stimulation to tissue of a brain of the patient or sense a patient parameter from a location within the blood vessel. The plurality of electrodes includes a first group of electrodes and a second group of electrodes such that when the distal portion of the elongated body is in the deployed configuration, each electrode of the first group of electrodes faces a first direction and each electrode of the second group of electrodes faces a second direction different from the first direction. The plurality of conductors includes a first conductor electrically coupled to each electrode of the first group of electrodes and a second conductor electrically coupled to each electrode of the second group of electrodes.

In some examples, an endovascular device includes a frame, a plurality of electrodes disposed along the frame, and a plurality of conductors. The frame is configured to expand from a relatively low-profile delivery configuration to an expanded configuration in a blood vessel of a patient. The plurality of electrodes is configured to deliver electrical stimulation to tissue of a brain of the patient or sense a patient parameter from a location within the blood vessel. The plurality of electrodes includes a first group of electrodes, a second group of electrodes, and a third group of electrodes wherein each electrode of the first group of electrodes faces a first direction, each electrode of the second group of electrodes faces a second direction different from the first direction, and each electrode of the third group of electrodes faces a third direction different from the first direction and the second direction. The plurality of conductors includes a first conductor electrically coupled to each electrode of the first group of electrodes, a second conductor electrically coupled to each electrode of the second group of electrodes, and a third conductor electrically coupled to each electrode of the third group of electrodes. The first conductor, the second conductor, and the third conductor form the frame.

In some examples, a method includes detecting, via processing circuitry configured to control therapy delivery via a plurality of electrodes disposed along an elongated body, a level of endothelization proximate the plurality of electrodes based on a sensed patient parameter. In some examples, the method further includes selecting, via the processing circuitry, one or more electrical stimulation parameter values based on detecting the level of endothelization proximate the plurality of electrodes. The elongated body is configured to be introduced in a blood vessel of a patient, and the plurality of electrodes is configured to deliver electrical stimulation to tissue of a brain of the patient or sense a patient parameter from a location within the blood vessel. The plurality of electrodes includes a first group of electrodes and a second group of electrodes, wherein each electrode of the first group of electrodes faces a first direction and each electrode of the second group of electrodes faces a second direction different from the first direction. A first conductor is electrically coupled to each electrode of the first group of electrodes and a second conductor is electrically coupled to each electrode of the second group of electrodes. Each electrode of the plurality of electrodes is a segmented electrode or a partial ring electrode.

The examples described herein may be combined in any permutation or combination.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating an example therapy system including an endovascular device configured to deliver electrical stimulation therapy to a target tissue site in a brain of a patient.

FIG. 2 is a functional block diagram illustrating components of an example endovascular device of the therapy system of FIG. 1.

FIG. 3A is a side view of an example distal portion of an elongated body of an example endovascular device.

FIG. 3B is a diagram illustrating an example electrode configuration of an example endovascular device.

FIG. 4 is a perspective view of the endovascular device shown in FIGS. 3A and 3B.

FIG. 5A is a side view of an example distal portion of an elongated body of an example endovascular device in a relatively low-profile delivery configuration.

FIG. 5B is a side view of the example distal portion of FIG. 5A in a deployed configuration.

FIG. 6 is a side view of an example frame of an example endovascular device.

FIG. 7 is a flow chart illustrating an example method of delivering therapy via an example endovascular device.

FIG. 8A illustrates example electrodes with insulative material covering a portion of the electrodes.

FIG. 8B illustrates a cross section of an example electrode from FIG. 8A.

Like reference characters denote like elements throughout the description and figures.

### DETAILED DESCRIPTION

This disclosure describes devices, systems, and methods relating to deep brain stimulation (DBS), such as delivery of electrical stimulation therapy to one or more deep brain stimulation (DBS) targets from an endovascular location. Example endovascular locations that can be used to access the DBS sites using the devices described herein include any suitable cranial vein or cranial artery, such as, but not limited to, the thalamostriate vein, the internal cerebral vein, the basal vein of Rosenthal, the inferior sagittal sinus, the superior sagittal sinus, or the anterior choroidal artery.

DBS has been proposed for use to manage one or more patient conditions, such as to treat a patient condition by reducing or even eliminating one or more symptoms associated with the patient condition. For example, DBS can be used to alleviate, and in some cases, eliminate symptoms associated with movement disorders, other neurodegenerative impairment, seizure disorders, psychiatric disorders (e.g., mood disorders), or the like. Movement disorders may be found in patients with Parkinson's disease, multiple sclerosis, and cerebral palsy, among other conditions, and can be associated with disease or trauma. DBS can be delivered to one or more target sites in a brain of a patient to help a patient with muscle control and minimize movement problems, such as rigidity, bradykinesia (i.e., slow physical movement), rhythmic hyperkinesia (e.g., tremor), nonrhythmic hyperkinesia (e.g., tics) or akinesia (i.e., a loss of physical movement).

In the case of seizure disorders, DBS can be delivered to one or more target sites in a brain of a patient to reduce the frequency or severity of seizures, or even help prevent the occurrence of seizures. In the case of psychiatric disorders, DBS can be delivered to help minimize or even eliminate symptoms associated with major depressive disorder (MDD), bipolar disorder, anxiety disorders, post-traumatic stress disorder, dysthymic disorder, or obsessive-compulsive disorder (OCD).

In the examples described herein, an endovascular device includes an elongated body (e.g., a catheter shaft) or a frame and a plurality of electrodes disposed on the elongated body or frame. The plurality of electrodes includes groups of electrodes that face in the same direction (e.g., a direction facing radially outward from the elongated body), which may provide a uniform or directional electrical stimulation field and/or facilitate directional sensing. The directional electrical stimulation field can help target certain regions of brain while minimizing delivery of electrical stimulation to other regions of brain associated with adverse effects. A group (e.g., electrodes that face the same direction) of electrodes includes two or more electrodes. Each electrode of a group of electrodes that faces the same direction or is otherwise radially separated from the electrodes of a different group of electrodes are electrically coupled to a common electrical conductor, such that the groups of electrodes that face the same direction are shorted or ganged together. Shorting or ganging groups of electrodes together via common conductors may reduce the number of conductors needed in the device, which may reduce the overall profile and complexity of the device.

In some examples, the elongated body or frame of the endovascular device includes one or more deployed configurations (e.g., coiled or expanded) that enables the electrodes disposed on the elongated body to be held in apposition with a blood vessel wall. Additionally, in some examples, the endovascular device includes one or more surface textures or coatings configured to promote endothelization around the electrodes or the device. Endothelization may reduce the overall power needed to deliver efficacious electrical stimulation therapy to a patient, as well as help secure the electrodes in place in the blood vessel in instances of chronic therapy delivery.

In some examples, the endovascular device includes processing circuitry configured to detect a level of endothelization proximate the plurality of electrodes and select (e.g., adjust) one or more electrical stimulation parameter values based on detecting the level of endothelization. Example electrical stimulation parameters include one or more or a power, amplitude, frequency, electrode combination, etc. The endovascular device can be configured to automatically select the one or more stimulation parameter values (e.g., without clinician intervention), which may promote consistent and effective therapy.

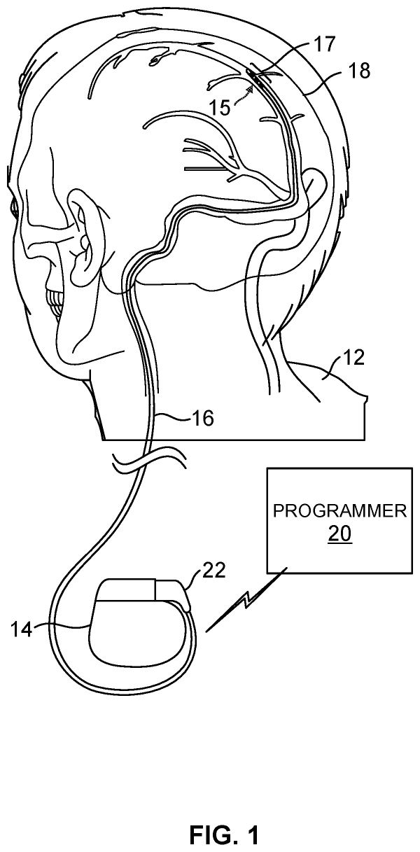

FIG. 1 is a conceptual diagram illustrating an example therapy system 10 configured to deliver electrical stimulation therapy to a target tissue site in a brain 18 of patient 12. Patient 12 ordinarily will be a human patient. In some cases, however, therapy system 10 may be applied to other mammalian or non-mammalian non-human patients. In some examples, therapy system 10 includes medical device 14, an endovascular device 16, and a plurality of electrodes 17 disposed on a distal portion 15 of endovascular device 16. In the example shown in FIG. 1, medical device 14 is configured to deliver DBS to brain 18 of patient 12 and/or sense bioelectrical brain signals in brain 18 via plurality of electrodes 17 of endovascular device 16. Endovascular device 16 is positioned in cranial vasculature of patient 12, such that plurality of electrodes 17 are located proximate to a target tissue site within brain 18 and are positioned to deliver electrical stimulation therapy to deep brain sites within brain 18 and/or sense one or more patient parameters, such as tissue sites under the dura mater surrounding brain 18. In some examples, placement of endovascular device 16, distal portion 15, and plurality of electrodes 17 is coincident with the dura mater, such as in the middle meningeal artery (MMA). Medical device 14 can provide electrical stimulation to one or more regions within brain 18 in order to manage a condition of patient 12, such as to mitigate the severity or duration of the patient condition, and/or sense one or more patient parameters to provide data and/or feedback required for managing a condition of the patient 12.

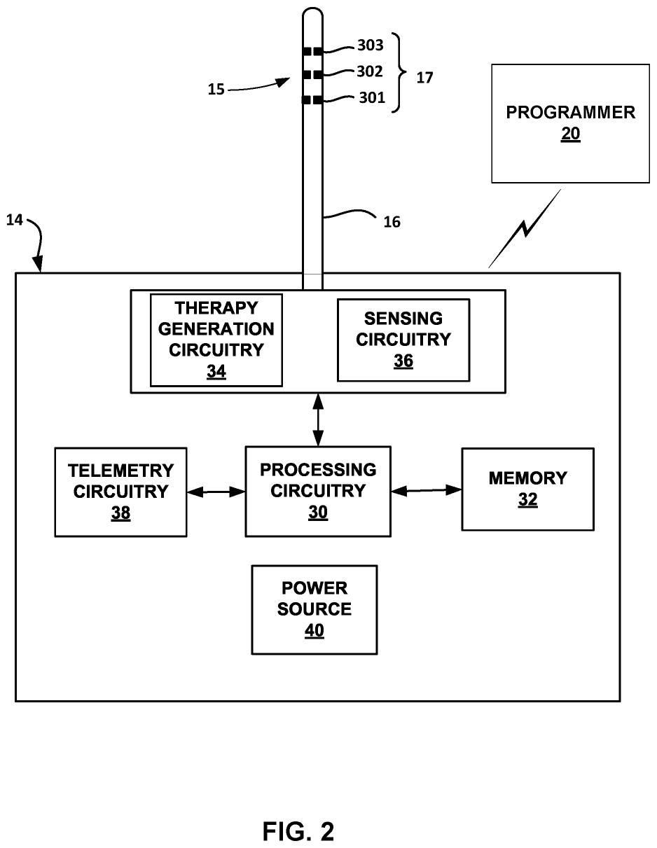

Endovascular device 16 includes any elongated body configured to deliver electrical stimulation signals to, and/or sense one or more patient parameters from, tissue proximate plurality of electrodes 17. For example, endovascular device 16 can be a medical lead, a catheter, a guidewire, or another elongated body carrying plurality of electrodes 17 and configured to be electrically coupled to medical device 14 either directly or indirectly via an electrically conductive pathway that runs between medical device 14 and plurality of electrodes 17. Endovascular device 16 has any suitable length that enables connection to medical device 14 either directly or indirectly, e.g., a length of 150 centimeters (cm) to 250 cm, such as 200 cm. As another example, endovascular device 16 can be a wireless therapy delivery device, such as a microstimulator or the like, which is not electrically coupled to medical device 14 via a wired connection. In some of these wireless therapy delivery device examples, system 10 does not include medical device 14 and endovascular device 16 includes therapy generation circuitry and/or other elements of medical device 14 described herein, e.g., with respect to FIG. 2. In some of these wireless therapy delivery device examples, distal portion 15 including plurality of electrodes 17 is configured to detach (e.g., via a detachment mechanism) from an elongated deliver member (e.g., a push wire or a hypotube) used to deliver distal portion 15 to a target site. Example detachment mechanisms can include, but are not limited to, those used to detach embolization devices from delivery devices, including those described in U.S. Pat. No. 8,328,860, entitled, “IMPLANT INCLUDING A COIL AND A STRETCH-RESISTANT MEMBER,” the disclosure of which is hereby incorporated herein by reference in its entirety.

In some examples, more than one endovascular device 16 may be positioned within brain 18 of patient 12 to provide stimulation to, and/or sense one or more patient parameters from, multiple anatomical regions of brain 18. Endovascular device 16 can be implanted in a blood vessel for chronic therapy delivery (e.g., on the order of months or even years) or for more temporary therapy delivery (e.g., on the order of days, such as less than a month or less than 6 months). In some examples, one or more devices (e.g., one or more of endovascular device 16) are placed in the MMA to provide stimulation and/or sense in corresponding regions of the brain, such as in the cortex. In some examples, one or more endovascular devices are placed within intracranial venous structures to provide electrical stimulation and/or sense in corresponding regions of the brain. When endovascular devices are placed in different regions of the brain, for example within multiple arterial locations, multiple venous locations, or within arterial and venous locations (e.g., MMA and deep venous system), the combined sensing from both modalities may provide temporal and spatial data. The temporal and spatial data can be used to control delivery of electrical stimulation therapy to patient 12 and/or to evaluate a patient condition at one point in time or over a longer time period.

DBS may be used to treat various patient conditions, such as, but not limited to, seizure disorders (e.g., epilepsy), pain, migraine headaches, psychiatric disorders (e.g., obsessive compulsive disorder, mood disorders or anxiety disorders), movement disorders (e.g., essential tremor or Parkinson's disease), Huntington's disease, and other neurodegenerative disorders. The anatomic region within brain 18 of patient 12 that serve as the target tissue site for electrical stimulation delivered by medical device 14 may be selected based on the patient condition. For example, stimulating an anatomical region, such as the substantia nigra, in brain 18 may reduce the number and magnitude of tremors experienced by patient 12. Other example target anatomical regions for treatment of movement disorders may include the subthalamic nucleus, globus pallidus interna, ventral intermediate, and zona inserta. Anatomical regions such as these may be targeted by the clinician during implantation of endovascular device 16. In other words, the clinician may attempt to position endovascular device 16 within or proximate to these target regions within brain 18 by positioning endovascular device 16 in a cranial blood vessel that is within or proximate to these target regions.

In various examples described herein, example regions of brain 18 that can include the target tissue site for electrical stimulation or sensing via endovascular device 16 positioned in a blood vessel in brain 18 include, but are not limited to, one or more of the anterior thalamus, the ventrolateral thalamus, the subthalamic nucleus (STN), the substantia nigra pars reticulata, the internal segment and/or external segments of the globus pallidus, the ventral intermediate, the zona inserta, the hippocampus (HIP), the dentate gyrus, the cortex (e.g., the motor strip, the sensor strip, the premotor cortex), the fornix, the neostriatum, the ventral intermediate nucleus of the thalamus, the cingulate, or the cingulate gyrus.

The vasculature into which endovascular device 16 may be inserted and/or guided includes, but is not limited to, veins or arteries. For example, to reach certain deep brain tissue sites, endovascular device 16 can be navigated from a vasculature access site (e.g., in the femoral artery, the radial artery, femoral vein, subclavian vein, internal jugular vein or another suitable access site) to one or more arterial structures (including, but not limited to the MMA) or veins of the superficial and a deep venous system (including, but not limited to the thalamostriate vein, the internal cerebral vein, the basal vein of Rosenthal, the inferior/superior sagittal sinus, the anterior choroidal artery, or any related combinations thereof).

Certain intracranial blood vessels into which endovascular device 16 may be inserted and/or guided may be located at different distances from different target tissue sites. Such distances may play a role in efficacy of therapy delivered by endovascular device 16, as a closer distance may indicate a shorter distance any electrical stimulation signal may have to travel, and, in some examples, the less power that is needed to generate an efficacious electrical stimulation signal. For example, the thalamostriate vein may be approximately 1.2 millimeters (mm) in diameter and be located approximately 0-2 mm from the anterior nucleus of the thalamus (ANT) and 0-2 mm from the fornix. As another example, the internal cerebral vein may be 1.9 mm plus or minus up to 0.5 mm in diameter and be located approximately 5-10 mm from the ANT and approximately 2-5 mm from the fornix. The basal vein of Rosenthal may be 1.7 mm plus or minus up to 0.4 mm in diameter and be located approximately 10-15 mm from the ANT, approximately 5-10 mm from the HIP, and approximately 5-10 mm from the STN. The inferior sagittal sinus may be 1.3 mm plus or minus up to 0.3 mm in diameter and be located approximately 10-15 mm from the Fornix.

A clinician can also select a particular intracranial blood vessel to position plurality of electrodes 17 at different orientations or distances relative to tissue sites (along with selectively activating groups of electrodes that face a certain direction) for which it may be desirable to avoid electrical stimulation to minimize or even eliminate adverse effects. DBS may cause one or more side effects by inadvertently providing electrical stimulation to anatomical regions near the targeted anatomical region. For this reason, a clinician may position plurality of electrodes 17 within brain 18 and/or program the electrical stimulation parameters in order to balance effective therapy and minimal side effects.

As discussed in further detail below, in some examples, endovascular device 16 is configured to be delivered to one or more target sites in brain 18 via vasculature of patient 12. Thus, rather than introducing endovascular device 16 into brain tissue (e.g., the cerebral parenchyma) via a burr hole through a skull of patient 12 or the like, endovascular device 16 is configured to be navigated to a target electrical stimulation site in brain 18 via vasculature of patient 12. The endovascular delivery of endovascular device 16 to deep brain sites in brain 18 can help minimize the invasiveness of therapy system 10.

In some examples, as discussed below, plurality of electrodes 17 are positioned on a portion of endovascular device 16 that is configured to deploy (e.g., expand) radially outwards from a relatively low-profile delivery configuration to a deployed (e.g., coiled or expanded) configuration. This may enable plurality of electrodes 17 to be held in apposition with a blood vessel wall, which may promote tissue ingrowth or endothelization around plurality of electrodes 17 along the vessel wall (while still leaving a patent lumen). Tissue ingrowth or endothelization around plurality of electrodes 17 can reduce the overall power needed to deliver efficacious electrical stimulation therapy to a target tissue site of brain 18, and help secure plurality of electrodes 17 in place in the blood vessel for chronic therapy delivery.

In some examples, plurality of electrodes 17 and or endovascular device 16 can include one or more surface textures or coatings to promote endothelization, decrease impedance, reduce thrombosis, or increase longevity. Materials used may include one or more of Titanium Nitride, Platinum, or poly(3,4-ethylenedioxythiophene) polystyrene sulfonate (“PEDOT:PSS” or “PDOT”). The one or more surface textures or coatings may increase a surface area of plurality of electrodes 17, which can help stabilize the impedance over a range of frequencies (e.g., of an electrical stimulation signal).

Medical device 14 can be an external medical device or an implantable medical device that includes electrical stimulation circuitry configured to generate and deliver electrical stimulation therapy to patient 12 via plurality of electrodes 17 of endovascular device 16. Plurality of electrodes 17 may be configured to deliver electrical stimulation to tissue of brain 18 of patient 12 from a location within a blood vessel. In the example shown in FIG. 1, endovascular device 16 is coupled to medical device 14 via connector 22, which defines a plurality of electrical contacts for electrically coupling plurality of electrodes 17 to electrical stimulation generation circuitry within medical device 14. Connector 22 may also be referred to as a connector block or header of medical device 14. In some examples, endovascular device 16 is indirectly coupled to connector 22 with the aid of a lead extension. In some examples, endovascular device 16 is directly coupled to connector 22 without the aid of a lead extension.

In some examples, medical device 14 is configured to be implanted in patient 12 in any suitable location, such as a location outside of brain 18, e.g., in a pectoral region. In other examples, medical device 14 is configured to be external to patient 12. Endovascular device 16 may be, for example, implanted within a cranial vein and one or more proximal wires/leads can remain within the venous system until they exit the subclavian vein in the chest for implant in the pectoral region. In yet other examples, some or all of medical device 14 is configured to be implanted in brain 18, e.g., as part of endovascular device 16.

As shown in FIG. 1, system 10 may also include a programmer 20, which may be a handheld device, portable computer, or workstation that provides a user interface to a clinician or other user. The clinician may interact with the user interface to program electrical stimulation parameters for medical device 14.

With the aid of programmer 20 or another computing device, a clinician may select values for therapy parameters for controlling therapy delivery by therapy system 10. The values for the therapy parameters may be organized into a group of parameter values referred to as a “therapy program” or “therapy parameter set.” “Therapy program” and “therapy parameter set” are used interchangeably herein. In the case of electrical stimulation, the therapy parameters may include an electrode combination, a power, and an amplitude, which may be a current or voltage amplitude, and, if medical device 14 delivers electrical pulses, a pulse width, and a pulse rate or frequency for stimulation signals to be delivered to the patient. Other example therapy parameters include a slew rate, duty cycle, and phase of the electrical stimulation signal. An electrode combination may include a selected group (e.g., electrodes that face the same direction) or subset (e.g., less than all of the electrodes) of plurality of electrodes 17 located on one or more implantable elongated bodies (such as endovascular device 16) coupled to medical device 14. The electrode combination may also refer to the polarities of the electrodes in the selected subset. By selecting particular electrode combinations, a clinician may target particular anatomic structures within brain 18 of patient 12. In addition, by selecting values for slew rate, duty cycle, phase amplitude, pulse width, and/or pulse rate, the clinician can attempt to generate an efficacious therapy for patient 12 that is delivered via the selected electrode subset.

Whether programmer 20 is configured for clinician or patient use, programmer 20 may communicate with medical device 14 or any other computing device via wireless or a wired communication. Programmer 20, for example, may communicate via wireless communication with medical device 14 using radio frequency (RF) telemetry techniques known in the art. Programmer 20 may also communicate with another programmer or computing device via a wired or wireless connection using any of a variety of local wireless communication techniques, such as RF communication according to the 802.11 or Bluetooth specification sets, infrared communication according to the Infrared Data Association (IRDA) specification set, or other standard or proprietary telemetry protocols. Programmer 20 may also communicate with another programming or computing device via a wired or wireless communication technique.

In some examples, in addition to or instead of delivering electrical stimulation to brain 18, endovascular device 16 can be used to sense one or more patient parameters, such as bioelectrical signals, either using plurality of electrodes 17 or other types of sensors that are carried by endovascular device 16. In some examples, the bioelectrical signals sensed within brain 18 reflect changes in electrical current produced by the sum of electrical potential differences across brain tissue. Examples of bioelectrical brain signals that can be sensed via one or more electrodes of plurality of electrodes 17 include, but are not limited to, electrical signals generated from local field potentials within one or more regions of brain 18, an electroencephalogram (EEG) signal, an electrocorticogram (ECoG) signal, or an evoked potential. In some examples, a sensing parameter includes one or more of a direction faced by each sensing electrode or group of electrodes (e.g., the active electrodes with which a medical device senses a patient parameter), a location of one or more electrodes within brain 18, or other parameters that may affect detection and/or sensing of one or more patient parameters.

Brain 18 in FIG. 1 is supplied with blood through the carotid and the vertebral arteries on each side of the neck. The arteries include the common carotid artery in the neck, which is a common access pathway for the various devices and/or methods disclosed herein, the internal carotid which supplies the ophthalmic artery. The external carotid supplies the maxillary artery, the middle meningeal artery (MMA), and the superficial temporal arteries (frontal and parietal). The vertebral artery supplies the basilar artery and the cerebral arteries including the posterior cerebral artery and the circle of Willis. The siphon of the vertebral artery appears in the intra-cranial vasculature on the vertebral approach to the Circle of Willis. Also supplied by the internal carotid artery are the anterior cerebral artery and the middle cerebral artery, as well as the circle of Willis, including the posterior communicating artery and the anterior communicating artery. The siphon of the internal carotid artery appears in the intra-cranial vasculature on the carotid approach into the Circle of Willis. These arteries can have an internal diameter of about 1 mm to 5 mm, most commonly from 2-4 mm.

The devices, systems, and methods described herein enable endovascular delivery to deep brain tissue sites in brain 18. Endovascular device 16 can be navigated to the cranial vasculature to reach the deep brain tissue sites, e.g., via an insertion catheter (e.g., a microcatheter). As an example, endovascular device 16 can be delivered to an intracranial blood vessel inside of a 0.017 inch (about 0.43 mm) or a 0.021 inch (about 0.53 mm) microcatheter, alone or with the aid of a guidewire.

In addition to, or instead of, chronic therapy delivery and/or chronic sensing, example devices, systems, and methods described herein can be used for more temporary applications. In some examples, a first endovascular device (e.g., configured like endovascular device 16 or having another configuration) is configured to be operated in an acute (e.g., temporary) trial mode for a trial period to determine, evaluate, or confirm an efficacy of stimulation and/or sensing. For example, endovascular device 16 may be configured to operate in the trial mode to determine the efficacy of one or more stimulation parameter values and/or one or more sensing parameters. After the acute trial period, the first endovascular device may be removed, and a second endovascular device (e.g., configured like endovascular device 16 or having another configuration) configured to operate in a chronic mode may be implanted for a chronic period for chronic (e.g., long term, or permanent) stimulation therapy or sensing. In some examples, a first endovascular device (e.g., for use in the acute trial mode) is configured to be implanted and subsequently removed after the trial period.

A trial period has a shorter intended duration than a chronic period, though the ultimate length of the chronic period may be less than an intended duration due to one or more factors, such as a patient response that requires shortening the chronic period relative to the intended duration of the chronic period. In some examples, the trial period includes a trial period length on the order of minutes (e.g., 1 minute, 2 minutes, 3 minutes, 5 minutes, 30 minutes, 45 minutes, etc.), on the order of hours (e.g., 1 hour, 2 hours, 5 hours, 12 hours, etc.), on the order of days (e.g., 1 day, 2 days, 3 days, etc.), on the order of weeks (e.g., 1 week, 2 weeks, 3 weeks, etc.) on the order of months (e.g., 1 month, 2 months, 3 months, etc.), or longer. In some examples, one or more endovascular devices may be used for multiple trial periods (e.g., successive trial periods) for determining an efficacy of one or more stimulation parameters and/or one or more sensing parameters.

In some examples, the trial period length is selected based on an endothelization profile. The endothelization profile may include a length of time for at least part of an endovascular device to become incorporated (or at least partially incorporated) into a vessel wall via tissue ingrowth or the like. For example, the length of the trial period may be selected such that the trial period is shorter than a period for an endovascular device (e.g., endovascular device 16) to become incorporated into a vessel wall via endothelization. The length of the trial period may be selected such that trial period is shorter than a period for the endovascular device to reach a threshold level of endothelization. The length of the trial period may correspond to a predetermined profile of endothelization (e.g., based on a model of endothelization, which can be patient specific or more general to a population of patients).

In some examples, the first endovascular device (e.g., for use in the acute trial mode) does not include features to promote endothelization. This may enable a clinician to remove the first endovascular device after the trial period with minimal impacts to a vessel wall (e.g., a portion of a vessel wall adjacent the endovascular device).

In some examples, the first endovascular device (e.g., the endovascular device used for the trial period) is configured to transform from a deployed configuration to a relatively low-profile configuration (as described above) to facilitate removal from a blood vessel of patient 12 after the trial period. For example, the first endovascular device (e.g., endovascular device 16) may be configured to recaptured, re-sheathed, or otherwise retracted (e.g., into a catheter or other outer sheath) to facilitate removal from patient 12 after the trial period. In examples in which the endovascular device includes the elongated body or frame configured to deploy (e.g., expand), the elongated body or the frame may be collapsed (e.g., via a sheath, pullwire, or another suitable method) to facilitate removal of the endovascular device from the vasculature of patient 12.

After determining or confirming the efficacy (e.g., the efficacy of one or more stimulation parameters and/or one or more sensing parameters) of the first endovascular device during the trial period, a second endovascular device (e.g., endovascular device 16) may be implanted and configured to operate in the chronic mode. In some examples, the second endovascular device is configured to deliver stimulation or sense a patient parameter in the chronic mode based on feedback from the trial period. For example, one or more therapy parameter values, as described above, are selected and/or changed based on feedback (e.g., one or more sensed patient parameters, feedback from patient 12 regarding the effects of therapy, and/or other indicators of the efficacy of therapy delivery) from the trial period.

As another example, one or more sensing parameters is selected and/or changed for the chronic period based on feedback from the trial period. In some examples, one or more implant locations within the brain is selected based on feedback from the trial period. In some examples, an orientation (e.g., a direction of one or more electrodes 17 facing outward from endovascular device 16) of the endovascular device is selected based on feedback from the trial period. In some examples, the second endovascular device includes one or more deployable configurations, surface textures or coatings, as described above, to promote endothelization around at least a portion of the second endovascular device.

While the second endovascular device may be provided for chronic therapy delivery or sensing, the first endovascular device (e.g., the endovascular device used for the trial period) may also be used during the chronic period. In some examples, a second endovascular device is not provided for the chronic period if the first endovascular device is used for the chronic period. In some examples, the first endovascular device is moved or repositioned for the chronic period. For example, endovascular 16 may be moved or reposition within the vasculature of patient 12 for the chronic period. The first endovascular device may be configured to operate in the chronic mode.

In some examples, the first endovascular device (e.g., the endovascular device used for the trial period) and the second endovascular device (e.g., the endovascular device used for the chronic period) may have different configurations of leads, electrodes, or operating parameters. As an example, the first endovascular device may include more electrodes as compared to the second endovascular device, which can provide more granular sensing or stimulation to enable a clinician to better identify therapy and/or sensing targets in brain 18. In some examples, the first endovascular device includes fewer electrodes than the second endovascular device. However, in some examples, the first endovascular device includes the same number of electrodes as compared to the second endovascular device.

In some examples, the same medical device 14 (e.g., including an electrical stimulation generation circuitry and/or sensing circuitry) is used for both the trial and chronic periods. In other examples, different medical devices are used for the trial and chronic periods. For example, the first endovascular device may be configured to electrically and mechanically connect to an external medical device configured to generate stimulation therapy and/or sense a patient parameter (e.g., bioelectrical brain signals) via electrodes of the first endovascular device and the second endovascular device may include a medical device (e.g., medical device 14) configured to be implanted in patient 12. In some cases, the external medical device can be configured similarly to medical device 14, but may not be configured for implantation in patient 12. In other examples, the external medical device can include fewer features than medical device 14, e.g., less memory, less sophisticated control circuitry. In addition, in some examples, the external medical device can be pre-programmed (e.g., by a clinician or a manufacturer) to deliver electrical stimulation therapy according to only a preselected group of therapy programs. In contrast, in some cases, a clinician programs medical device 14 used for chronic therapy delivery to deliver efficacious electrical stimulation therapy 14 to patient 14 or effective sensing of one or more parameters of patient 14 during a programming period.

FIG. 2 is a functional block diagram illustrating components of an example medical device 14, which is configured to generate and deliver electrical stimulation therapy to patient 12 and, in some examples, sense one or more patient parameters, such as bioelectrical brain signals of patient 12. Medical device 14 includes processing circuitry 30, memory 32, therapy generation circuitry 34, sensing circuitry 36, telemetry circuitry 38, and power source 40.

Therapy generation circuitry 34 includes any suitable configuration (e.g., hardware) configured to generate electrical stimulation signals to a target tissue site in brain 18 of patient 12. Processing circuitry 30 is configured to control therapy generation circuitry 34 to generate and deliver electrical stimulation therapy via plurality of electrodes 17 (shown as clusters of electrodes 301, 302, and 303 in the example of FIG. 2) of endovascular device 16. Plurality of electrodes 17 may include a monopolar or bipolar arrangement. The electrical stimulation parameter values may be selected based on the patient condition being addressed, as well as the target tissue site in brain 18 for the electrical stimulation therapy. The electrical stimulation therapy can be provided via stimulation signals of any suitable form, such as stimulation pulses or continuous-time signals (e.g., sine waves).

Sensing circuitry 36 is configured to sense a physiological parameter of a patient. Sensing circuitry 36 may include any sensing hardware configured to sense a physiological parameter of a patient, such as, but not limited to, one or more electrodes, optical receivers, pressure sensors, or the like. The one or more sensing electrodes can be the same or different from plurality of electrodes 17 configured to deliver electrical stimulation therapy. Processing circuitry 30 can use the sensed physiological signals to control therapy delivery by therapy generation circuitry 34, e.g., the timing of the therapy delivery or one or more characteristics of the electrical simulation signal generated by therapy generation circuitry 34.

In some examples, sensing circuitry 36 is configured to sense a bioelectrical brain signal via plurality of electrodes 17 (e.g., all or a subset of electrodes 17). Thus, plurality of electrodes 17 can be configured to receive or transmit energy (e.g., current). Example bioelectrical brain signals include an EEG signal, an ECoG signal, a signal generated from measured field potentials within one or more regions of brain 18, action potentials from single cells within brain 18 (referred to as “spikes”), or evoked potentials. Determining action potentials of single cells within brain 18 may require resolution of bioelectrical signals to the cellular level and provides fidelity for fine movements, i.e., a bioelectrical signal indicative of fine movements (e.g., slight movement of a finger). In examples in which endovascular device 16 is configured to sense an evoked potential, endovascular device 16 may also be configured to generate a stimulus (e.g., via therapy generation circuitry, alone or in combination with processing circuitry 30) to elicit the evoked potential. For example, endovascular device 16 can generate and deliver electrical stimulation to tissue in brain 18 and sense an evoked compound action potential (ECAP). An ECAP is synchronous firing of a population of neurons which occurs in response to the application of a stimulus including, in some cases, an electrical stimulus by endovascular device 16. The ECAP may be detectable as being a separate event from the stimulus itself, and the ECAP may reveal characteristics of the effect of the stimulus on the tissue.

In some examples, processing circuitry 30, alone or in combination with sensing circuitry 36, is configured to determine a level of endothelization proximate one or more of the electrodes 17 based on a sensed parameter. The parameter is any suitable parameter that can be sensed by processing circuitry 30 via electrodes 17 or another sensor and that changes as a function of the level of endothelization and can include, for example, impedance of an electrical pathway that includes the one or more electrodes 17. In some examples, a level of endothelization proximate plurality of electrodes 17 includes the extent to which endothelial cells cover endovascular device 16 and/or one or more electrodes 17, which may affect the impedance of the respective electrodes 17. The level can include a level at a particular point in time or a change in a level of endothelization over time, e.g., relative to a previously determined level, such as a baseline level at the time of implantation of endovascular device 16 in patient 12. For example, the change in a level of endothelization (e.g., an altered impedance) can be a result of sensing a level of impedance of one or more electrodes of the plurality of electrodes 17 at a first time point and at a second time point, and calculating a change in the impedance level between the first time point and the second time point.

In some examples, processing circuitry 30, alone or in combination with therapy generation circuitry 34, is configured to select one or more electrical stimulation parameter values based on a determined level of endothelization or in response to detecting a change in the level of endothelization proximate plurality of electrodes 17. As described above, the electrical stimulation parameters (or “therapy parameters”) may include an electrode combination (e.g., selective activation of a group or subset of electrodes from plurality of electrodes 17), a power, and an amplitude, which may be a current or voltage amplitude, and, if medical device 14 delivers electrical pulses, a pulse width, and a pulse rate or frequency for stimulation signals to be delivered to the patient. Other example electrical stimulation parameters include a slew rate, duty cycle, and phase of the electrical stimulation signal. In some examples, processing circuitry 30, alone or in combination with therapy generation circuitry 34, is configured to automatically select or adjust one or more electrical stimulation parameter values (e.g., without clinician intervention) or may otherwise provide a prompt or recommendation to select or adjust one or more electrical stimulation parameter values (e.g., such as via programmer 20). In some examples, a sensed level of endothelization is utilized along with patient factors (e.g., symptoms, side effects, or other sensed parameters) to select or adjust one or more stimulation parameters. Endothelization proximate endovascular device 16 and or plurality of electrodes 17 may facilitate the ability to provide stimulation therapy or sense patient parameters, which may enhance the fidelity of therapy. In some examples, when a sufficient level of endothelization has been reached, a need for anticoagulation therapy (e.g., commonly dual antiplatelet therapy (DAPT)) is reduced or eliminated.

In some examples, sensing circuitry 36 and/or processing circuitry 30 includes signal processing circuitry configured to perform any suitable analog conditioning of the sensed physiological signals. For example, sensing circuitry 36 may communicate to processing circuitry 30 an unaltered (e.g., raw) signal. Processing circuitry 30 may be configured to modify a raw signal to a usable signal by, for example, filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof. In some examples, the conditioned analog signals may be processed by an analog-to-digital converter of processing circuitry 30 or other component to convert the conditioned analog signals into digital signals. In some examples, processing circuitry 30 may operate on the analog or digital form of the signals to separate out different components of the signals. In some examples, sensing circuitry 36 and/or processing circuitry 30 may perform any suitable digital conditioning of the converted digital signals, such as low pass, high pass, band pass, notch, averaging, or any other suitable filtering, amplifying, performing an operation on the signal, performing any other suitable digital conditioning, or any combination thereof. Additionally or alternatively, sensing circuitry 36 may include signal processing circuitry to modify one or more raw signals and communicate to processing circuitry 30 one or more modified signals.

Although shown as part of medical device 14 in FIG. 2, in other examples, sensing circuitry 36 can be a part of a device separate from medical device 14. For example, sensing circuitry 36 can be part of an implantable sensing device implanted in cranial vasculature or elsewhere in brain 18 of patient 12.

Processing circuitry 30, as well as other processors, processing circuitry, controllers, control circuitry, and the like, described herein, may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, control circuitry may include multiple components, such as any combination of one or more microprocessors, one or more DSPs, one or more ASICs, or one or more FPGAs, as well as other discrete or integrated logic circuitry, and/or analog circuitry.

Memory 32 is configured to store program instructions, such as software, which may include one or more program modules, which are executable by processing circuitry 30. When executed by processing circuitry 30, such program instructions may cause processing circuitry 30 to provide the functionality ascribed to processing circuitry 30 herein. The program instructions may be embodied in software and/or firmware. Memory 32 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Processing circuitry 30 is configured to control telemetry circuitry 38 to send and receive information. Telemetry circuitry 38, as well as telemetry modules in other devices described herein, such as programmer 20, may accomplish communication by any suitable communication techniques, such as RF communication techniques. In addition, telemetry circuitry 38 may communicate with programmer 20 via proximal inductive interaction of medical device 14 with programmer 20. Accordingly, telemetry circuitry 38 may send information to programmer 20 on a continuous basis, at periodic intervals, or upon request from medical device 14 or programmer 20.

Power source 40 is configured to deliver operating power to various components of medical device 14. Power source 40 may include a small rechargeable or non-rechargeable battery and a power generation circuit to produce the operating power. Recharging may be accomplished through proximal inductive interaction between an external charger and an inductive charging coil within medical device 14. In some examples, power requirements may be small enough to allow medical device 14 to utilize patient motion and implement a kinetic energy-scavenging device to trickle charge a rechargeable battery. In other examples, traditional batteries may be used for a limited period of time.

As discussed above, in some examples, endovascular device 16 is configured to be a standalone electrical stimulation device and can include one or more elements of medical device 14 shown in FIG. 2.

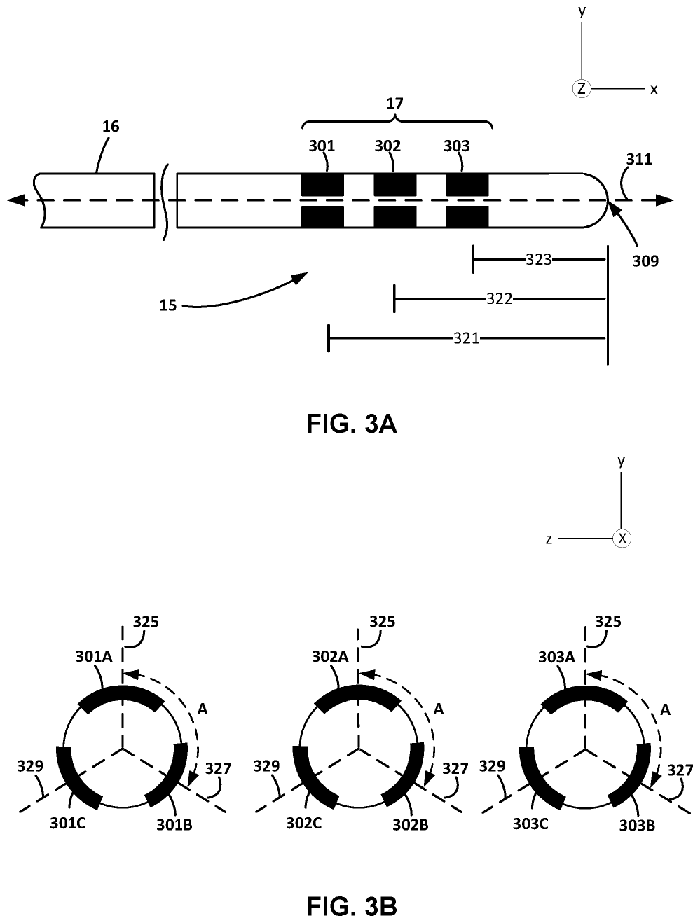

FIGS. 3A and 3B illustrate an example distal portion 15 of an example endovascular device 16, which may be a medical lead, and plurality of electrodes 17. FIG. 3A shows a two-dimensional side view in the x-y plane of endovascular device 16, which includes three clusters of electrodes 301, 302, and 303. Within each cluster, two or more (e.g., two, three, four or more) electrodes are longitudinally aligned and each cluster of electrodes is disposed at a different longitudinal distance from a distal-most end 309 of endovascular device 316 along longitudinal axis 311. Thus, endovascular device 16 includes a plurality of longitudinally distributed clusters of electrodes 301, 302, 303. FIG. 3B shows a cross-sectional view in the y-z plane of each of the three clusters of electrodes 301, 302, and 303 through the longitudinal center of each the three clusters of electrodes 301, 302, and 303. That is, the cross-sectional views shown in FIG. 3B correspond to cross-sections of endovascular device 16 taken through a longitudinal center of the respective cluster of electrodes and in a direction orthogonal to longitudinal axis 311 of endovascular device 16. The orthogonal x-y-z axes used in the figures are used to aid the description of the figures.

In the example of FIGS. 3A and 3B, each cluster of electrodes 301, 302, and 303 includes three segmented electrodes (shown in FIG. 3B as 301A, 301B, 301C, 302A, 302B, 302C, 303A, 303B, and 303C) distributed around an outer perimeter (e.g., an outer circumference in the case of a circular cross-section) of endovascular device 16. Each segmented (or partial ring) electrode only extends partially around the outer perimeter endovascular device 16. In other examples, endovascular device 16 may include any number and combination of clusters of partial ring electrodes or segmented electrodes. For example, endovascular device 16 may include only one cluster of segmented electrodes (e.g., for monopolar stimulation or sensing), but can also include two, three, or more clusters. As another example, each respective cluster of electrodes 301, 302, and 303 may include more than three segmented (or partial ring) electrodes, but may also include one or two segmented or partial ring electrodes. In some examples, each cluster of electrodes 301, 302, and 303 has the same number of electrodes, but each cluster of electrodes 301, 302, and 303 may have a different number of electrodes.

FIG. 3B illustrates groups of electrodes of the plurality of electrodes 17 that face a same direction (e.g., a direction facing radially outward from endovascular device 16, or in reference to FIG. 3A, a particular direction radially outward from longitudinal axis 311, rather than 360 degrees around longitudinal axis 311). For example, each electrode of a first group of electrodes 301A/302A/303A faces a first direction 325 (e.g., a first direction extending radially outward from a center of endovascular device 16). Similarly, each electrode of a second group of electrodes 301B/302B/303B faces a second direction 327 (e.g., a second direction extending radially outward from a center of endovascular device 16) that is different from the first direction 325. Similarly, each electrode of a third group of electrodes 301C/302C/303C faces a third direction 329 (e.g., a third direction extending radially outward from a center of endovascular device 16) that is different from the first direction 325 and the second direction 327. Directions 325, 327, 329 can have any suitable spacing from each other about longitudinal axis 311, as discussed below with respect to angle A and can be evenly distributed about longitudinal axis 311 or unevenly distributed in other examples, e.g., if deliver of electrical stimulation in a particular direction is desired.

In the example of FIGS. 3A and 3B, each electrode of a group of electrodes are circumferentially aligned around endovascular device 16, but radially separated from other groups of electrodes. For example, as shown in the example of FIG. 3B, each electrode of first group of electrodes 301A/302A/303A is circumferentially aligned around endovascular device 16, but first group of electrodes 301A/302A/303A is radially separated from second group of electrodes 301B/302B/303B by an angle A around endovascular device 16. In some examples, angle A is 120 degrees, or about 120 degrees (to the extent permitted by manufacturing tolerances). In some examples, angle A equals 360 degrees divided by the total number of groups of electrodes.

In the example of FIG. 3B, first group of electrodes 301A/302A/303A is also radially separated from third group of electrodes 301C/302C/303C by angle A around endovascular device 16. Similarly, each electrode of a second group of electrodes 301B/302B/303B is circumferentially aligned around endovascular device 16, but second group of electrodes 301B/302B/303B is radially separated from first group of electrodes 301A/302A/303A by angle A around endovascular device 16 and also radially separated from third group of electrodes 301C/302C/303C by angle A around endovascular device 16. Similarly, each electrode of a third group of electrodes 301C/302C/303C is circumferentially aligned around endovascular device 16, but third group of electrodes 301C/302C/303C is radially separated from first group of electrodes 301A/302A/303A by angle A around endovascular device 16 and also radially separated from second group of electrodes 301B/302B/303B by angle A around endovascular device 16. In this way, first group of electrodes 301A/302A/303A, second group of electrodes 301B/302B/303B, and third group of electrodes 301C/302C/303C are uniformly spaced around endovascular device 16. More particularly, electrodes of a given cluster of electrodes 301/302/303 are uniformly spaced around endovascular device 16 (e.g., electrode 301A, electrode 301B, and electrode 301C are separated from each other by angle A). However, in other examples, the groups of electrodes can be unevenly distributed about the longitudinal axis 211 such that angle A is different for different sets of circumferentially adjacent groups of electrodes. That is, first group of electrodes 301A/302A/303A, second group of electrodes 301B/302B/303B, and third group of electrodes 301C/302C/303C may not be uniformly spaced around endovascular device 16, and may be radially separated by any suitable degree around endovascular device 16. For example, first group of electrodes 301A/302A/303A can be closer to second group of electrodes 301B/302B/303B than to third group of electrodes 301C/302C/303C.

In the example of FIG. 3A, clusters of electrodes are disposed at different longitudinal distances from a distal-most end 309 of endovascular device 316 along longitudinal axis 311. For example, a first cluster of electrodes 301 (which includes a first electrode 301A of first group of electrodes 301A/302A/303A, a first electrode 301B of second group of electrodes 301B/302B/303B, and a first electrode 301C of third group of electrodes 301C/302C/303C) is disposed at a first distance 321 along longitudinal axis 311 from distal-most end 309 of endovascular device 316 to a longitudinal center of first cluster of electrodes 301. A second cluster of electrodes 302 (which includes a second electrode 302A of first group of electrodes 301A/302A/303A, a second electrode 302B of second group of electrodes 301B/302B/303B, and a second electrode 302C of third group of electrodes 301C/302C/303C) is disposed at a second distance 322 along longitudinal axis 311 from distal-most end 309 of endovascular device 316 to a longitudinal center of second cluster of electrodes 302. A third cluster of electrodes 303 (which includes a third electrode 303A of first group of electrodes 301A/302A/303A, a third electrode 303B of second group of electrodes 301B/302B/303B, and a third electrode 303C of third group of electrodes 301C/302C/303C) is disposed at a third distance 323 along longitudinal axis 311 from distal-most end 309 of endovascular device 316 to a longitudinal center of third cluster of electrodes 303.

In some examples, first distance 321 is different than the second distance 322 and the third distance 323. In some examples second distance 322 is different than the first distance 321 and the third distance 323. In some examples third distance 323 is different than the first distance 321 and the second distance 322. In some examples, clusters of electrodes 301, 302, and 303 are uniformly spaced along longitudinal axis 311 of elongate body 316 (e.g., each cluster is spaced from an adjacent cluster by a given longitudinal distance, such as 0.5 millimeters, 1 millimeter, 2 millimeters, 3 millimeters, or more). In some examples, clusters of electrodes 301, 302, and 303 are not uniformly spaced along longitudinal axis 311 of elongate body 316. In the example of FIG. 3B, each electrode is separated from a center of endovascular device 16 (e.g., from a longitudinal axis 311) by a uniform distance.

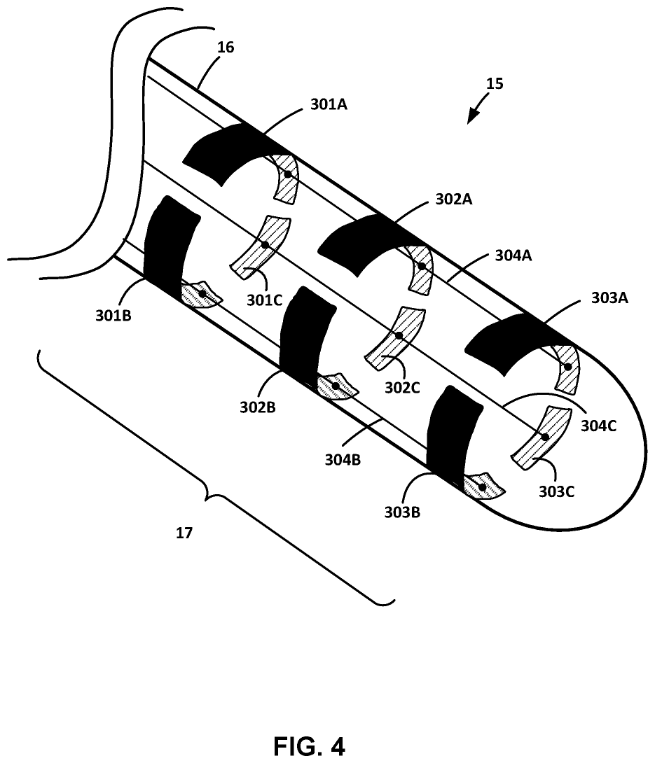

FIG. 4 illustrates a perspective view of the distal portion 15 of the example endovascular device 16 from FIG. 3A, which includes a plurality of electrical conductors 304 (shown individually as first conductor 304A, second conductor 304B, and third conductor 304C) electrically coupled to the plurality of electrodes 17. In the example of FIG. 4, a first conductor 304A is electrically coupled to first group of electrodes 301A/302A/303A, a second conductor 304B is electrically coupled to second group of electrodes 301B/302B/303B, and a third conductor 304C is electrically coupled to third group of electrodes 301C/302C/303C. First conductor 304A is not electrically connected to second group of electrodes 301B/302B/303B or third group of electrodes 301C/302C/303C. Second conductor 304B is not electrically connected to first group of electrodes 301A/302A/303A or third group of electrodes 301C/302C/303C. Third conductor 304C is not electrically connected to first group of electrodes 301A/302A/303A or second group of electrodes 301B/302B/303B.

In this example, each electrode of first group of electrodes 301A/302A/303A is shorted or ganged together, each electrode of second group of electrodes 301B/302B/303B is shorted or ganged together, and each electrode of third group of electrodes 301C/302C/303C is shorted or ganged together. In this way, processing circuitry 30, alone or in combination with therapy generation circuitry 34, can independently activate first group of electrodes 301A/302A/303A, second group of electrodes 301B/302B/303B, and third group of electrodes 301C/302C/303C, via first conductor 304A, second conductor 304B, or third conductor 304C respectively. For example, processing circuitry 30, alone or in combination with therapy generation circuitry 34, can be configured to deliver electrical stimulation or sense via first group of electrodes 301A/302A/303A without delivering electrical stimulation via second group of electrodes 301B/302B/303B or third group of electrodes 301C/302C/303C. Similarly, sensing circuitry 36 may be configured to receive independent signals via first group of electrodes 301A/302A/303A, second group of electrodes 301B/302B/303B, and third group of electrodes 301C/302C/303C.

Because groups of electrodes that face the same direction (e.g., first group of electrodes 301A/302A/303A that face a first direction, second group of electrodes 301B/302B/303B that face a second direction, and third group of electrodes 301C/302C/303C that face a third direction) are shorted or ganged together via the plurality of conductors 304, processing circuitry 30, alone or in combination with therapy generation circuitry 34 may selectively activate multiple electrodes that face the same direction and sensing circuitry 36 may selectively sense from multiple electrodes that face the same direction. By controlling the directionality of electrode activation, the resulting directional electrical stimulation field can help target certain regions of brain 18 (FIG. 1) while minimizing delivery of electrical stimulation to other regions of brain associated with adverse effects. Where certain electrodes or the plurality of electrodes 17 are configured to sense a patient parameter, groups of electrodes that face the same direction may allow for directional sensing (e.g., sensing in only a certain direction less than 360 degrees around endovascular device 16). This can help, for example, processing circuitry 30 identify a target stimulation site in brain 18, identify non-target anatomical region (to avoid stimulating), determine the effect of electrical stimulation therapy that was delivered, determine a rotational orientation (about longitudinal axis 311 of FIG. 3A) of distal portion 15 within brain 18, sense activity within specific anatomical regions of brain 18 (e.g., to detect patient events, such as one or more of a seizures, a speech state, a sleep state, an ischemic state, a movement state, or any change in a bioelectrical brain signal (e.g., EEG, ECoG, or evoked potentials) corresponding to a specific patient event or physiological state of interest), and the like.

Electrodes 17 can have any suitable segmented or partial ring configuration. In some examples, some or all of plurality of electrodes 17 are integrally formed with at least distal portion 15 of endovascular device 16. For example, at least distal portion 15 of endovascular device 16 is formed from an electrically conductive material (e.g., one or more nitinol core wires) that is electrically connected to therapy generation circuitry 34 and sensing circuitry 36 (FIG. 2) and an electrically insulative material can be positioned radially outwards of the electrically conductive material to cover the electrically conductive material. In such examples, where endovascular device 16 includes an electrically insulative material, at least a portion of the electrically insulative material is removed to expose the electrically conductive material to define plurality of electrodes 17. To define plurality of electrodes 17, part of the electrically insulative material can be removed (e.g., via laser ablation, mechanical etching, or the like) to expose the electrically conductive material. Any suitable electrically insulative material can be used, such as, but not limited to, polytetrafluoroethylene (PTFE), expanded PTFE (ePTFE), silicone, polyimide, non-metallic oxide, parylene or the like. The electrically insulative material can have any suitable thickness, such as, but not limited to, 0.010 mm to 0.05 mm (e.g., about 0.0005 inches).

In other examples, plurality of electrodes 17 are or include a component physically separate from endovascular device 16 and mechanically connected to the endovascular device 16. For example, plurality of electrodes 17 includes an electrically conductive electrode material (e.g., platinum, tungsten, gold, or the like, which can be radiopaque or not) electrically coupled to one or more electrically conduct materials extending through endovascular device 16 (e.g., plurality of conductors 304).

Although the examples of FIG. 3 and FIG. 4 show each group of electrodes that are shorted or ganged together and face a common direction (e.g., first group of electrodes 301A/302A/303A, second group of electrodes 301B/302B/303B, and third group of electrodes 301C/302C/303C), as having three electrodes, groups of electrodes may have any suitable number of electrodes (e.g., one, two, three, four, or more electrodes). In some examples, groups of electrodes may include the same number of electrodes (e.g., each group has three electrodes). However, in other examples, groups of electrodes include a different number of electrodes (e.g., a first group of electrodes includes three electrodes, while a second group of electrodes and a third group of electrodes includes two electrodes). Further, while the examples of FIG. 3 and FIG. 4 show three groups of electrodes, any suitable number of different groups of electrodes may be disposed on endovascular device 16 (e.g., one, two, three, four, or more groups of electrodes).

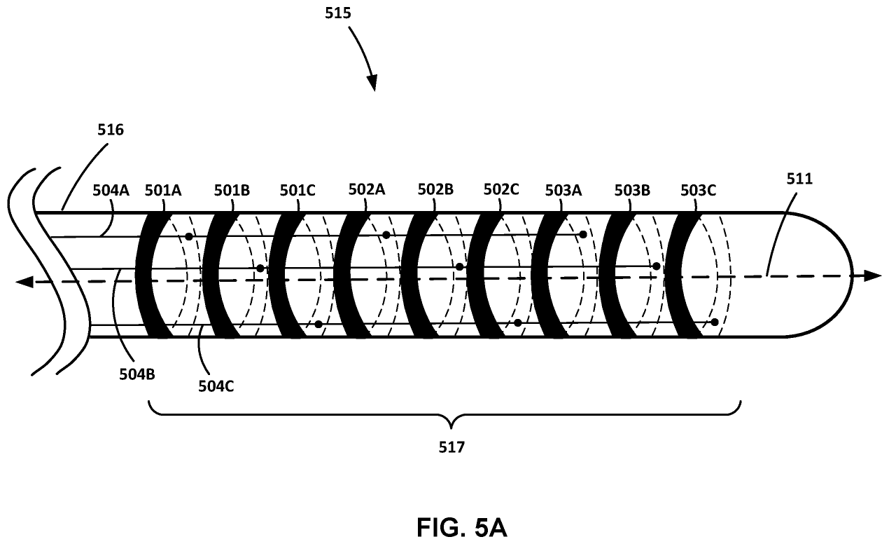

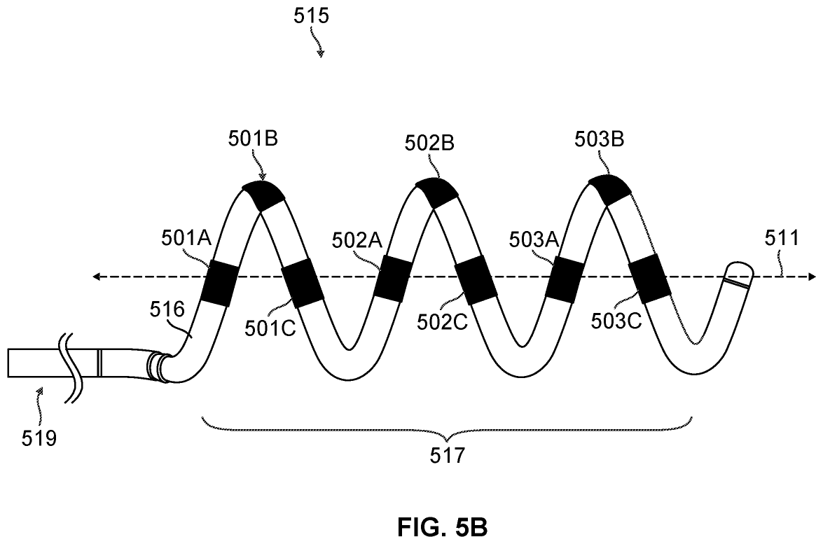

FIGS. 5A-5B illustrate another example distal portion 515 of an example endovascular device 516, which may be a medical lead, and plurality of electrodes 517 disposed along the endovascular device 516. Endovascular device 516 and plurality of electrodes 517 are examples of endovascular device 16 and electrodes 17 of FIGS. 1-4 and may be the same as those as described in relation to FIGS. 1-4 except as noted herein. In the example of FIGS. 5A and 5B, distal portion 515 is transformable between a relatively low-profile delivery configuration to facilitate delivery through vasculature to a target tissue site in a brain (e.g., FIG. 5A) and a deployed configuration (e.g., FIG. 5B) in which the distal portion 515 is expanded to position plurality of electrodes 517 in apposition with a blood vessel wall. In the example shown in FIG. 5B, distal portion 515 defines a coil in the deployed configuration (which may also be referred to as the “expanded” configuration). The deployed configuration illustrated in FIG. 5B may also include a helical configuration or a spiral configuration.

In some examples, distal portion 515 of endovascular device 516 includes a shape memory (e.g., nitinol) material that enables distal portion 515 to assume a predetermined shape in the absence of a force (e.g., a compressive or tensile force) holding the distal portion 515 in a relatively low-profile delivery configuration. For example, distal portion 515 can be configured to expand radially outwards upon deployment from an outer sheath (e.g., an outer catheter), or upon the proximal withdrawal of a straightening element (e.g., a guidewire or a mandrel) positioned in an inner lumen of endovascular device 516. As another example, the distal portion 515 can be configured to expand radially outwards in response to proximal withdrawal of a pull member attached to distal portion 515 of the endovascular device 516 or in response to a distal movement of an elongated control member attached to distal portion 515.

FIG. 5A illustrates a perspective view of an example distal portion 515 of an endovascular device 516 in the low-profile delivery configuration, and illustrates a plurality of electrical conductors 504 (shown individually as first conductor 504A, second conductor 504B, and third conductor 504C) electrically coupled to the plurality of electrodes 517 (shown individually as electrode 501A, 501B, 501C, 502A, 502B, 502C, 503A, 503B, and 503C). Specifically, in the example of FIG. 5A, a first conductor 504A is electrically coupled to first group of electrodes 501A/502A/503A, a second conductor 504B is electrically coupled to second group of electrodes 501B/502B/503B, and a third conductor 504C is electrically coupled to third group of electrodes 501C/502C/503C. First conductor 504A is not electrically connected to second group of electrodes 501B/502B/503B or third group of electrodes 501C/502C/503C. Second conductor 504B is not electrically connected to first group of electrodes 501A/502A/503A or third group of electrodes 501C/502C/503C. Third conductor 504C is not electrically connected to first group of electrodes 501A/502A/503A or second group of electrodes 501B/502B/503B.

In this example, each electrode of first group of electrodes 501A/502A/503A is shorted or ganged together, each electrode of second group of electrodes 501B/502B/503B is shorted or ganged together, and each electrode of third group of electrodes 501C/502C/503C is shorted or ganged together. In this way, processing circuitry 30, alone or in combination with therapy generation circuitry 34, can independently activate first group of electrodes 501A/502A/503A, second group of electrodes 501B/502B/503B, and third group of electrodes 501C/502C/503C via first conductor 504A, second conductor 504B, or third conductor 504C respectively. For example, processing circuitry 30, alone or in combination with therapy generation circuitry 34, can be configured to deliver electrical stimulation or sense via first group of electrodes 501A/502A/503A without delivering electrical stimulation via second group of electrodes 501B/502B/503B or third group of electrodes 501C/502C/503C. Similarly, sensing circuitry 36 may be configured to receive independent signals via first group of electrodes 501A/502A/503A, second group of electrodes 501B/502B/503B, and third group of electrodes 501C/502C/503C.

In the example of FIG. 5A, each electrode of the plurality of electrodes 517 extend fully around a perimeter (e.g., an outer circumference in the case of a circular cross-section) of endovascular device 516 (e.g., a ring electrode). However, in some examples, plurality of electrodes 517 can include one or more partial ring or segmented electrodes, wherein at least some or all electrodes of the plurality of electrodes 517 extend only partially around a perimeter of endovascular device 516. In some examples, plurality of electrodes 517 includes a combination of at least one ring electrode and at least one or more partial ring or segmented electrodes. The combination and orientation of plurality of electrodes 517 may be chosen based on the target location for therapy delivery, and the type of therapy being delivered, as well as other suitable reasons.

In the example of FIG. 5A, where distal portion 515 in a relatively low-profile delivery configuration, distal portion 515 may be straight or substantially straight such that each electrode of the plurality of electrodes is aligned along endovascular device 516 (e.g., a center of each electrode of the plurality of electrodes 517 is approximately aligned along a central longitudinal axis 511 of endovascular device 516). In the example of FIG. 5A, when distal portion 515 is in the relatively low-profile delivery configuration, plurality of electrodes 517 extends along endovascular device 516 in the following order (proximally to distally): 501A, 501B, 501C, 502A, 502B, 502C, 503A, 503B, and 503C. In some examples, each electrode of the plurality of electrodes 517 may be uniformly spaced from corresponding adjacent electrodes of the plurality of electrodes 517 (e.g., where the same longitudinal distance separates a center of each electrode along endovascular device 516). However, in some examples, each electrode of the plurality of electrodes 517 may not be uniformly spaced from corresponding adjacent electrodes of the plurality of electrodes 517 (e.g., where the different longitudinal distances separate centers of electrodes along endovascular device 516).

FIG. 5B illustrates a side view of an example distal portion 515 of an endovascular device 516 in the deployed (e.g., expanded) configuration, which in the example of FIG. 5B is a configuration in which distal portion 515 is expanded and defines a coil around central longitudinal axis 511. In some examples, distal portion 515 is configured to expand radially outwards with sufficient force and to a cross-sectional dimension (e.g., a diameter) sufficient to position the plurality of electrodes 517 in apposition with a blood vessel wall. This may help promote tissue ingrowth around plurality of electrodes 517, which can reduce the impedance and the overall power needed to deliver efficacious electrical stimulation therapy to a target tissue, and help secure plurality of electrodes 517 in place in the blood vessel for chronic (e.g., on the order of months or even years) therapy delivery. Fixing endovascular device 516 in place within the blood vessel via the tissue ingrowth or, in some examples, using another fixation structures/anchoring mechanisms, such as tines, coils barbs, or the like, can also help reduce the possibility of thrombosis.

In some examples, endovascular device 516 includes a proximal portion 519 which is proximal of distal portion 515. As shown in the example of FIG. 5B, proximal portion 519 may be manufactured in a straight (or relatively straight, as permitted by manufacturing tolerances) form factor, and remains straight even when distal portion 515 is deployed. However, in other examples, proximal portion 519 includes one or more deployed configuration (e.g., coiled or expanded) that will create apposition with a blood vessels wall. In some examples, at least a region of proximal portion 519 is configured to expand radially outwards (e.g., a coil or helix) with sufficient force and to a cross-sectional dimension (e.g., a diameter) sufficient to position the region of proximal portion 519 in apposition with a blood vessel wall. This may help promote tissue ingrowth around the region of proximal portion 519, which can reduce hydromechanical stresses on the region of proximal portion 519, help secure the region of proximal portion 519 in place in the blood vessel for chronic (e.g., on the order of months or even years) therapy delivery and reduce the likelihood of thrombus formation.

As shown in the example of FIG. 5B, each group of electrodes, including first group of electrodes 501A/502A/503A, second group of electrodes 501B/502B/503B, and third group of electrodes 501C/502C/503C, face a different direction when distal portion 515 is in the deployed (e.g., expanded) configuration. In the example of FIG. 5B, the different directions that each group of electrodes face includes a radial direction outward from a central longitudinal axis 511 that is faced by an outermost surface of each electrode of the plurality of electrodes 517. For example, each electrode of a first group of electrodes 501A/502A/503A faces a first direction (e.g., a first direction extending radially outward from central longitudinal axis 511). Similarly, each electrode of a second group of electrodes 501B/502B/503B faces a second direction (e.g., a second direction extending radially outward from central longitudinal axis 511) that is different from the first direction. Similarly, each electrode of a third group of electrodes 501C/502C/503C faces a third direction (e.g., a third direction extending radially outward from central longitudinal axis 511) that is different from the first direction and the second direction. Because each electrode of the respective groups of electrodes that face the same direction are shorted or ganged together via a common conductor (as discussed above with respect to FIG. 5A), processing circuitry 30, alone or in combination with therapy generation circuitry 34, may independently activate one or more groups of electrodes to selectively activate multiple electrodes that face the same direction. “Activate” can refer to, for example, delivering electrical stimulation or sensing a parameter via the respective group of electrodes. By controlling the directionality of electrode activation, the resulting directional electrical stimulation field can help target certain regions of brain 18 (FIG. 1) while minimizing delivery of electrical stimulation to other regions of brain 18 associated with adverse effects.

In the example of FIG. 5B, the coil defined by distal portion 515 when distal portion 515 is in the deployed configuration defines a relatively constant radius from central longitudinal axis 511 to a part of distal portion 515 that defines the coil along the longitudinal length of distal portion 515. However, in some examples, the coil defined by distal portion 515 when distal portion 515 is in the deployed configuration defines a changing radius from central longitudinal axis 511 to a part of distal portion 515. For example, in some examples, the coil defined by distal portion 515 of endovascular device 516 defines a spiral that tapers proximally or distally (e.g., wherein the coil changes in radius along central longitudinal axis 511 from a distal portion to a more proximal portion of distal portion 515 of endovascular device 516).

In the example of FIG. 5B, where distal portion 515 is in the deployed (e.g., expanded) configuration, only electrodes of a given group (e.g., each electrode of the first group of electrodes 501A/502A/503A, each electrode of the second group of electrodes 501B/502B/503B, and each electrode of the third group of electrodes 501C/502C/503C) are aligned along a direction parallel to central longitudinal axis 511. For example, electrode 501A, electrode 502A, and electrode 503A of the first group of electrodes 501A/502A/503A are aligned when distal portion 515 in the deployed (e.g., expanded) configuration, but are not aligned with any electrodes of the second group of electrodes 501B/502B/503B or the third group of electrodes 501C/502C/503C when distal portion 515 in the deployed (e.g., expanded) configuration.

Groups of electrodes 17, 517 on endovascular devices 16, 516 (which are shown as elongated bodies), respectively, are described above. In other examples, therapy system 10 can include electrodes on another structure, such as an expandable frame.

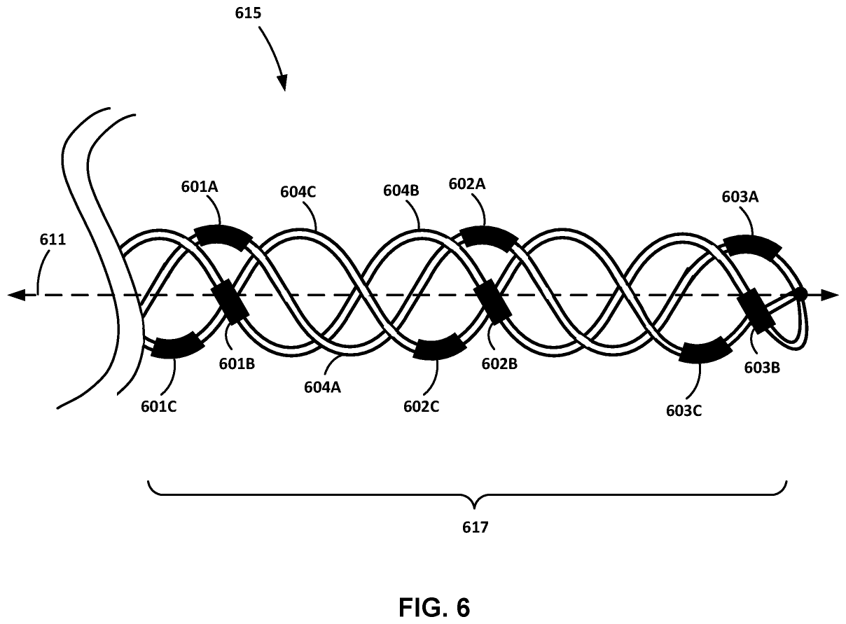

FIG. 6 is a side view of an example frame 615 including a plurality of electrodes 617 (shown individually as shown individually as electrode 601A, 601B, 601C, 602A, 602B, 602C, 603A, 603B, and 603C) disposed along frame 615, and a plurality of electrical conductors 604 (shown individually as first conductor 604A, second conductor 604B, and third conductor 604C) electrically coupled to the plurality of electrodes. In the example of FIG. 6, first conductor 604A, second conductor 604B, and third conductor 604C form frame 615. In other examples, frame 615 can be formed from one or more structures separate from conductors 604A, 604B, 604C.

In the example of FIG. 6, frame 615 is transformable between a relatively low-profile delivery configuration to facilitate delivery through vasculature to a target tissue site in brain 18 and a deployed configuration (also referred to as an expanded configuration) in which frame 615 is expanded to position plurality of electrodes 617 in apposition with a blood vessel wall. Frame 615 may be coupled to an elongated shaft (e.g., a catheter shaft), and may function similarly to distal portion 515 of endovascular device 516 as described in connection with FIGS. 5A and 5B, except as noted herein. In other examples, frame 615 may not be coupled to an elongated shaft, but, rather, can be delivered to a target site in vasculature of a patient using devices, systems, and methods similar to those used to implant stents or other expandable structures in vasculature of a patient. In some of these examples, conductors 604A, 604B, 604C may still extend from an implanted frame 615 in cranial vasculature to medical device 14. In some examples, at least some of conductors 604A, 604B, 604C are configured to expand (e.g., as a helix or a spiral) in a region proximal of plurality of electrodes 617 to position the conductors in apposition with a blood vessel wall. This may help promote tissue ingrowth around the conductors, which can reduce hydromechanical stresses on conductors 604A, 604B, 604C in that region, help secure the conductors in that region in place in the blood vessel for chronic (e.g., on the order of months or even years) therapy delivery and reduce the likelihood of thrombus formation.

In some examples, frame 615 includes a shape memory (e.g., nitinol) material that enables frame 615 to assume a predetermined shape in the absence of a force (e.g., a compressive or tensile force) holding frame 615 in a relatively low-profile delivery configuration. For example, the frame 615 can be configured to expand radially outwards upon deployment from an outer sheath (e.g., an outer catheter). As another example, frame 615 can be configured to expand radially outwards in response to an expansion force (e.g., from an expanding balloon) positioned within the interior of frame 615.

As noted above, first conductor 604A, second conductor 604B, and third conductor 604C form frame 615 in the example of FIG. 6. For example, first conductor 604A, second conductor 604B, and third conductor 604C may each define a coil (e.g., a helix or spiral) and may each wrap around a central longitudinal axis 611. Frame 615 formed by first conductor 604A, second conductor 604B, and third conductor 604C may generally define a tubular shape in the deployed configuration. Each conductor may be offset from adjacent conductors such that the electrodes of the plurality of electrodes 617 coupled to each conductor faces a different direction. In other examples, frame 615 can have another suitable structure, e.g., similar to a stent. For example, frame 615 can be formed from a cut hypotube and can define a plurality of interconnected struts.

In the example of FIG. 6, a first conductor 604A is electrically coupled to first group of electrodes 601A/602A/603A, a second conductor 604B is electrically coupled to second group of electrodes 601B/602B/603B, and a third conductor 604C is electrically coupled to third group of electrodes 601C/602C/603C. First conductor 604A is not electrically connected to second group of electrodes 601B/602B/603B or third group of electrodes 601C/602C/603C. Second conductor 604B is not electrically connected to first group of electrodes 601A/602A/603A or third group of electrodes 601C/602C/603C. Third conductor 604C is not electrically connected to first group of electrodes 601A/602A/603A or second group of electrodes 601B/602B/603B. In this example, each electrode of first group of electrodes 601A/602A/603A is shorted or ganged together, each electrode of second group of electrodes 601B/602B/603B is shorted or ganged together, and each electrode of third group of electrodes 601C/602C/603C is shorted or ganged together. In this way, processing circuitry 30, alone or in combination with therapy generation circuitry 34, can independently activate first group of electrodes 601A/602A/603A, second group of electrodes 601B/602B/603B, and third group of electrodes 601C/602C/603C via first conductor 604A, second conductor 604B, or third conductor 604C respectively. For example, processing circuitry 30, alone or in combination with therapy generation circuitry 34, can be configured to deliver electrical stimulation via first group of electrodes 601A/602A/603A without delivering electrical stimulation via second group of electrodes 601B/602B/603B or third group of electrodes 601B/602B/603B. Similarly, sensing circuitry 36 may be configured to receive independent signals via first group of electrodes 601A/602A/603A, second group of electrodes 601B/602B/603B, and third group of electrodes 601C/602C/603C.

In the example of FIG. 6, each electrode of the plurality of electrodes 617 extend fully around a perimeter (e.g., an outer circumference in the case of a circular cross-section) of each respective conductor of the plurality of conductors 604. However, in some examples, plurality of electrodes 617 can include one or more partial ring or segmented electrodes, wherein at least some electrodes of the plurality of electrodes 617 extend only partially around each respective conductor of the plurality of conductors 604. In some examples, plurality of electrodes 617 includes a combination of at least one ring electrode and at least one or more partial ring or segmented electrodes. The combination and orientation of plurality of electrodes 617 may be chosen based on the target location for therapy delivery, and the type of therapy being delivered, as well as other suitable reasons. In some examples, plurality of conductors 604 includes an electrically insulative material, and at least a portion of the electrically insulative material is removed to expose the electrically conductive material to define plurality of electrodes 617. However, in other examples, each electrode of the plurality of electrodes 607 is separate from and coupled to the respective conductor of the plurality of conductors 604.

In some examples, frame 615, is configured to expand radially outwards with sufficient force and to a cross-sectional dimension (e.g., a diameter) sufficient to position the plurality of electrodes 617 in apposition with a blood vessel wall. This may help promote tissue ingrowth around plurality of electrodes 617, which can reduce the impedance and the overall power needed to deliver efficacious electrical stimulation therapy to a target tissue, and help secure plurality of electrodes 617 in place in the blood vessel for chronic (e.g., on the order of months or even years) therapy delivery. Frame 615 may be fixed in place within the blood vessel via the tissue ingrowth or, in some examples, using another fixation structures/anchoring mechanisms, such as tines, coils barbs, or the like, can also help reduce the possibility of thrombosis.

As shown in the example of FIG. 6, each group of electrodes, including first group of electrodes 601A/602A/603A, second group of electrodes 601B/602B/603B, and third group of electrodes 601C/602C/603C, face a different direction when frame 615 is in the deployed configuration. In the example of FIG. 6, the different directions that each group of electrodes face includes a radial direction outward from a central longitudinal axis 611 that is faced by an outermost surface of each electrode of the plurality of electrodes 617. For example, each electrode of a first group of electrodes 601A/602A/603A faces a first direction (e.g., a first direction extending radially outward from central longitudinal axis 611). Similarly, each electrode of a second group of electrodes 601B/602B/603B faces a second direction (e.g., a second direction extending radially outward from central longitudinal axis 611) that is different from the first direction. Similarly, each electrode of a third group of electrodes 601C/602C/603C faces a third direction (e.g., a third direction extending radially outward from central longitudinal axis 611) that is different from the first direction and the second direction. Because each electrode of the respective groups of electrodes that face the same direction are shorted or ganged together via a common conductor (as discussed above), a user may selectively activate or sense from multiple electrodes that face the same direction. By controlling the directionality of electrode activation, the resulting directional electrical stimulation field can help target certain regions of brain while minimizing delivery of electrical stimulation to other regions of brain associated with adverse effects.

In the example of FIG. 6, the coil defined by each conductor of the plurality of conductors 604 defines a relatively constant radius along frame 615 from central longitudinal axis 611 to a part of each conductor of the plurality of conductors 604 (e.g., a center point of each conductor of the plurality of conductors 604). However, in some examples, the coil defined by each conductor of the plurality of conductors 604 may define a radius along frame 615 from central longitudinal axis 611 that changes along a length of frame 615 (e.g., such that each conductor of the plurality of conductors 604 is a spiral).

In the example of FIG. 6, when frame 615 is in the deployed configuration, only electrodes of a given group (e.g., each electrode of the first group of electrodes 601A/602A/603A, each electrode of the second group of electrodes 601B/602B/603B, and each electrode of the third group of electrodes 601C/602C/603C) are circumferentially aligned. For example, electrode 601A, electrode 602A, and electrode 603A of the first group of electrodes 601A/602A/603A are circumferentially aligned when frame 615 is in the deployed configuration, but are not circumferentially aligned with any electrodes of the second group of electrodes 601B/602B/603B or the third group of electrodes 601C/602C/603C when frame 615 is in the deployed configuration.

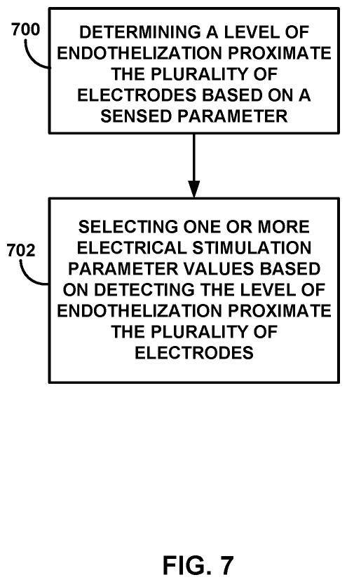

FIG. 7 is a flow diagram illustrating an example technique for delivering therapy via an endovascular device according to one or more examples of this disclosure. The technique in FIG. 7 may be used in connection with any of the devices or systems described in connection with FIGS. 1-6, and is described with respect to medical device 14, endovascular device 16, as well as the various devices and system described in FIGS. 1-4.

In some examples, the technique of FIG. 7 includes determining, via processing circuitry 30 (or, alternatively sensing circuitry 36 or therapy generation circuitry 34, alone or in any suitable combination with processing circuitry 30), a level of endothelization proximate the plurality of electrodes 17 based on a sensed parameter (700). For example, processing circuitry 30 (or, alternatively sensing circuitry 36 or therapy generation circuitry 34, alone or in any suitable combination with processing circuitry 30) may sense an impedance value of an electrical pathway including one or more electrodes of the plurality of electrodes 17, which may be the result of endothelization. As discussed above, a level of endothelization proximate plurality of electrodes 17 may include the extent to which tissue has grown around plurality of electrodes 17, which may affect the impedance of the plurality of electrodes 17. In some examples, processing circuitry 30 (or, alternatively sensing circuitry 36 or therapy generation circuitry 34, alone or in any suitable combination with processing circuitry 30) may detect a change in a level of endothelization proximate the plurality of electrodes 17 based on a sensed physiological parameter (e.g., a change in impedance of plurality of electrodes 17 over a time period). The change in a level of endothelization (e.g., an altered impedance) can be a result of sensing a level of impedance of one or more electrodes of the plurality of electrodes 17 at a first time point and at a second time point, and calculating a change in the impedance level between the first time point and the second time point.

Continuing with the example technique of FIG. 7, processing circuitry 30 (or, alternatively sensing circuitry 36 or therapy generation circuitry 34, alone or in any suitable combination with processing circuitry 30) selects one or more electrical stimulation parameter values based on detecting the level of endothelization proximate the plurality of electrodes 17 (702). In some examples, the electrical stimulation parameter includes one or more of a one or more of a frequency, a power, or an amplitude of electrical stimulation, but can also include other parameters as noted previously. For example, where plurality of electrodes 17 senses a certain level of in impedance proximate plurality of electrodes 17 (e.g., low, medium, or high), processing circuitry 30 may automatically select the frequency, power or amplitude values to an appropriate level (or initiate a prompt for a user to select electrical stimulation parameter values, such as via programmer 20). In some examples, the electrical stimulation parameter includes selective activation of a subset of electrodes from the plurality of electrodes, wherein the subset includes one or more groups of electrodes of the plurality of electrodes 17.

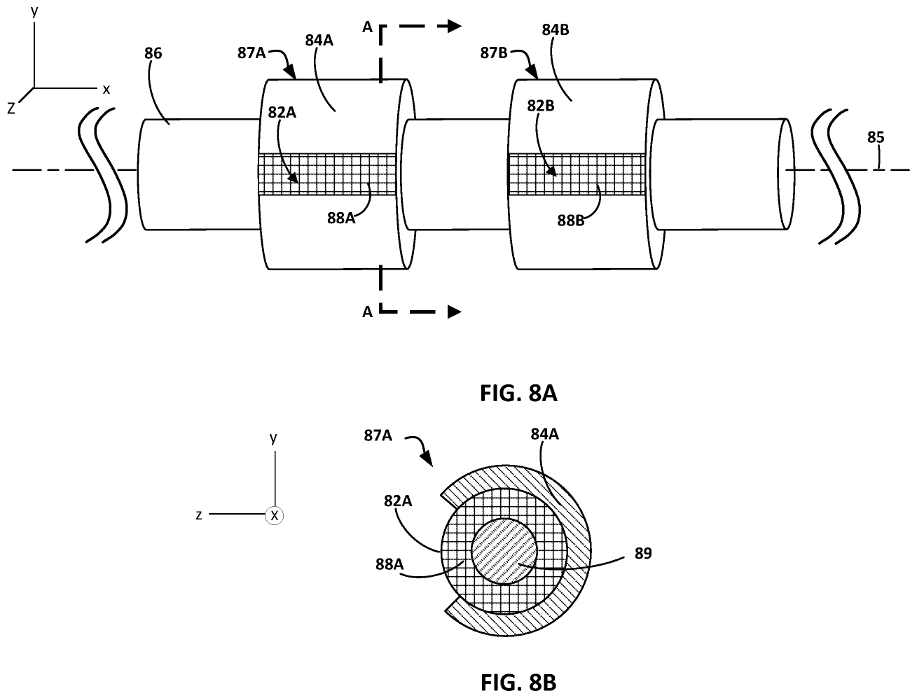

FIGS. 8A and 8B illustrate an example of how electrodes may be partially covered by electrically insulative material to further facilitate directional stimulation and/or sensing. In the example perspective view of FIG. 8A, an elongated body 86 includes electrodes 87A and 87B (collectively “electrodes 87”, which may be examples of electrodes 17, electrodes 517, and the like) spaced longitudinally along elongated body 86. Electrodes 87 may be spaced longitudinally along elongated body 86 along a central longitudinal axis 85 of elongated body 86 (extending in the x-axis direction in the example of FIGS. 8A and 8B, where orthogonal x-y-z axes are shown in FIGS. 8A and 8B for ease of description).

Electrode 87A includes electrically insulative material 84A surrounding at least a portion of an outer perimeter (e.g., outer diameter in examples in which electrode 87A has a circular cross-section) of an electrically conductive material 88A. Similarly, electrode 87B includes electrically insulative material 84B surrounding at least a portion of an outer perimeter (e.g., outer diameter) of an electrically conductive material 88B. Insulative material 84A and insulative material 84B (collectively “insulative material 84”) comprise a suitable electrically insulative material (e.g., a polymer), such that when insulative material 84 is positioned between conductive material 88A and conductive material 88B (collectively “conductive material 88”) and tissue of a patient, insulative material 84 reduces or even prevents conductive material 88 from transmitting electrical signals to or sensing signals from tissue of a patient.

In some examples, electrodes 87 include conductive material 88 at least partially covered by insulative material 84 such that only a section 82A and a section 82B (collectively “sections 82”) of conductive material 88 is exposed (e.g., exposed to blood and tissue within a blood vessel of patient 12). Sections 82 of exposed conductive material 88 may face in a direction (e.g., a direction radially outward from central longitudinal axis 85) to facilitate directional stimulation and/or sensing. Additionally, using sections 82 of exposed conductive material 88 on elongated body 86 may have an overall lower profile as compared to other alternatives. In the example of FIG. 8A, section 82 generally faces in the z-axis direction, which is radially outward from longitudinal axis 85 of elongated body 86. In use, elongated body 86 may be rotated to position section 82 adjacent a target location within the vasculature for stimulation and/or sensing via electrodes 87.

Sections 82 may be formed using any suitable method. In some examples, a method of forming includes covering conductive material 88 (e.g., an entire outer surface of conductive material 88) with electrically insulative material 84, and subsequently removing (e.g., ablating) portions of the electrically insulative material 84 to define sections 82. In some examples, electrically insulative material 84 is added to conductive material 88 such that when electrically insulative material 84 is added, a gap or opening in insulative material 84 defines sections 82.

Electrodes 87 may include a suitable number and orientations of exposed sections 82 of conductive material 88 (e.g., depending on the type of expandable element used, the target tissue site within brain 18, and the like). In some examples, each electrode of electrodes 87 includes more than one section 82 of exposed conductive material. In some examples, sections 82 of exposed conductive material 88 on elongated body 86 may act as segmented electrodes (in examples with multiple sections 82). In some examples, electrodes 87 include multiple sections 82 rotationally spaced apart along the circumference from each respective section of sections 82 (e.g., where each of electrode 87A and 87B include a first exposed section 82 generally facing the z-axis direction as shown in FIG. 8A, and another exposed section generally facing the y-axis direction). In some examples, exposed section 82A of electrode 87A and exposed section 82B of electrode 87B face in the same direction (e.g., the z-axis direction, as shown in the example of FIG. 8A). However, in some examples, exposed section 82A of electrode 87A and exposed section 82B of electrode 87B face in different directions (e.g., exposed section 82A faces the z-axis direction, and exposed section 82B faces in the y-axis direction).

FIG. 8B illustrates a cross section of an example elongated body and electrodes of FIG. 8A, the cross-section taken through a plane transverse (e.g., orthogonal) to the longitudinal axis 85 of elongated body 86 (section A-A indicated in FIG. 8A). As discussed in connection with FIG. 8A, electrode 87A includes insulative material 84A around an outer perimeter of electrode 87A. For example, insulative material 84A may cover (e.g., be radially outwards of) at least a portion of a conductive material 88A to define at least one exposed section 82A of conductive material 88A. In some examples, electrode 87A includes a core 89 radially inward from conductive material 88A and insulative material 84A. Core 89 may include another conductive material (e.g., the same material as conductive material 88, or more may include one or more wires (e.g., coated wires) that act as conductors for electrodes 87 e.g., to connect electrodes 87 to a medical device including electrical stimulation generation circuitry and/or sensing circuitry.

Although FIGS. 8A and 8B illustrate electrodes 87 as protruding (e.g., in the y-axis and z-axis directions) from an outermost surface of elongated body 86, in some examples, electrodes 87 are formed to be flush or about flush with the outermost surface of elongated body 86.

The techniques described in this disclosure, including those attributed to system 10, medical device 14, programmer 20, or various constituent components, may be implemented, at least in part, in hardware, software, firmware or any combination thereof. For example, various aspects of the techniques may be implemented within one or more processors, including one or more microprocessors, digital signal processors (DSPs), application specific integrated circuits (ASICs), field-programmable gate array (FPGAs), or any other equivalent integrated or discrete logic circuitry, as well as any combinations of such components, embodied in programmers, such as clinician or patient programmers, medical devices, or other devices. Processing circuitry, control circuitry, and sensing circuitry, as well as other processors and controllers described herein, may be implemented at least in part as, or include, one or more executable applications, application modules, libraries, classes, methods, objects, routines, subroutines, firmware, and/or embedded code, for example. In addition, analog circuits, components and circuit elements may be employed to construct one, some or all of the processing circuitry 30, instead of or in addition to the partially or wholly digital hardware and/or software described herein. Accordingly, analog or digital hardware may be employed, or a combination of the two.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. The computer-readable medium may be an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include random-access memory (RAM), read-only memory (ROM), programmable ROM (PROM), erasable programmable ROM (EPROM), electronically erasable programmable ROM (EEPROM), flash memory, a hard disk, a compact disc ROM (CD-ROM), a floppy disk, a cassette, magnetic media, optical media, or any other computer readable storage devices or tangible computer readable media.

In some examples, a computer-readable storage medium includes a non-transitory medium. The term “non-transitory” may indicate that the storage medium is not embodied in a carrier wave or a propagated signal. In certain examples, a non-transitory storage medium may store data that can, over time, change (e.g., in RAM or cache).

The functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements.

Example 1. An endovascular device comprising: an elongated body configured to be introduced in a blood vessel of a patient; a plurality of electrodes disposed along the elongated body, the plurality of electrodes comprising a first group of electrodes and a second group of electrodes; and a plurality of conductors comprising a first conductor electrically coupled to each electrode of the first group of electrodes and a second conductor electrically coupled to each electrode of the second group of electrodes, wherein each electrode of the first group of electrodes faces a first direction and each electrode of the second group of electrodes faces a second direction different from the first direction, wherein each electrode of the plurality of electrodes is a segmented electrode or a partial ring electrode, and wherein the plurality of electrodes is configured to deliver electrical stimulation to tissue of a brain of the patient or sense a patient parameter from a location within the blood vessel.

Example 2. The endovascular device of example 1, wherein the plurality of electrodes further comprises a third group of electrodes, wherein the plurality of conductors further comprises a third conductor electrically coupled to each electrode of the third group of electrodes, and wherein each electrode of the third group of electrodes faces a third direction different from the first direction and the second direction.

Example 3. The endovascular device of example 2, wherein the first group of electrodes, the second group of electrodes, and the third group of electrodes are independently activatable.

Example 4. The endovascular device of any of examples 2-3, wherein each electrode of the first group of electrodes is circumferentially aligned along the elongated body, wherein each electrode of the second group of electrodes is circumferentially aligned along the elongated body, and wherein each electrode of the third group of electrodes is circumferentially aligned along the elongated body.

Example 5. The endovascular device of any of examples 2-4, wherein the first group of electrodes is separated from the second group of electrodes and the third group of electrodes by 120 degrees around the elongated body.

Example 6. The endovascular device of any of examples 2-5, wherein the first group of electrodes comprises three or more electrodes, the second group of electrodes comprises three or more electrodes, and the third group of electrodes comprises three or more electrodes.

Example 7. The endovascular device of any of examples 2-6, wherein a first electrode of the first group of electrodes, a first electrode of the second group of electrodes, and a first electrode of the third group of electrodes are disposed on the elongated body at a first distance from a distal-most end of the elongated body, wherein a second electrode of the first group of electrodes, a second electrode of the second group of electrodes, and a second electrode of the third group of electrodes are disposed on the elongated body at a second distance from the distal-most end of the elongated body, and wherein a third electrode of the first group of electrodes, a third electrode of the second group of electrodes, and a third electrode of the third group of electrodes are disposed on the elongated body at a third distance from the distal-most end of the elongated body.

Example 8. The endovascular device of any of examples 1-7, further comprising a medical lead comprising the elongated body.

Example 9. An endovascular device comprising: an elongated body comprising a distal portion transformable between a relatively low-profile delivery configuration and a deployed configuration, wherein the distal portion is configured to be introduced in a blood vessel of a patient; a plurality of electrodes disposed along the elongated body, the plurality of electrodes comprising a first group of electrodes and a second group of electrodes; and a plurality of conductors comprising a first conductor electrically coupled to each electrode of the first group of electrodes and a second conductor electrically coupled to each electrode of the second group of electrodes, wherein when the distal portion of the elongated body is in the deployed configuration, each electrode of the first group of electrodes faces a first direction and each electrode of the second group of electrodes faces a second direction different from the first direction, and wherein the plurality of electrodes is configured to deliver electrical stimulation to tissue of a brain of the patient or sense a patient parameter from a location within the blood vessel.

Example 10. The endovascular device of example 9, wherein the plurality of electrodes further comprises a third group of electrodes, wherein the plurality of conductors further comprises a third conductor electrically coupled to each electrode of the third group of electrodes, and wherein each electrode of the third group of electrodes faces a third direction different from the first direction and the second direction.

Example 11. The endovascular device of any of examples 9-10, wherein in the relatively low-profile delivery configuration, each electrode of the plurality of electrodes is aligned along the elongated body.

Example 12. The endovascular device of any of examples 9-11, wherein each electrode of the plurality of electrodes is a segmented electrode or a partial ring electrode.

Example 13. The endovascular device of any of examples 1-12, wherein the elongated body comprises an electrically insulative material, wherein the at least a portion of the electrically insulative material is removed to expose an electrically conductive material to define the plurality of electrodes.

Example 14. An endovascular device comprising: a frame configured to expand from a relatively low-profile delivery configuration to a deployed configuration in a blood vessel of a patient; a plurality of electrodes disposed along the frame, the plurality of electrodes comprising a first group of electrodes, a second group of electrodes, and a third group of electrodes; and a plurality of conductors comprising a first conductor electrically coupled to each electrode of the first group of electrodes, a second conductor electrically coupled to each electrode of the second group of electrodes, and a third conductor electrically coupled to each electrode of the third group of electrodes, wherein the first conductor, the second conductor, and the third conductor form the frame, wherein each electrode of the first group of electrodes faces a first direction, each electrode of the second group of electrodes faces a second direction different from the first direction, and each electrode of the third group of electrodes faces a third direction different from the first direction and the second direction, and wherein the plurality of electrodes is configured to deliver electrical stimulation to tissue of a brain of the patient or sense a patient parameter from a location within the blood vessel.

Example 15. The endovascular device of example 14, wherein the frame comprises a shape memory material, and wherein the frame is expandable.

Example 16. The endovascular device of any of examples 1-15, wherein the plurality of electrodes comprises platinum, tungsten, or gold.

Example 17. The endovascular device of any of examples 1-16, wherein the endovascular device is configured to be operated in a trial mode for a trial period to determine an efficacy of the electrical stimulation or sensing.

Example 18. The endovascular device of any of examples 1-16, further comprising one or more surface textures configured to promote endothelization within the blood vessel.

Example 19. The endovascular device of any of examples 1-18, further comprising processing circuitry configured to control therapy delivery via the plurality of electrodes, wherein the processing circuitry is configured to detect a level of endothelization proximate the plurality of electrodes based on a sensed parameter.

Example 20. The endovascular device of example 19, wherein the sensed parameter is an impedance.

Example 21. The endovascular device of any of examples 19-20, wherein the processing circuitry is configured to select one or more electrical stimulation parameters values based on detecting the level of endothelization proximate the plurality of electrodes.

Example 22. The endovascular device of example 21, wherein one or more electrical stimulation parameters comprises one or more of a frequency, a power, or an amplitude of electrical stimulation.

Example 23. The endovascular device of any of examples 21-22, wherein one or more electrical stimulation parameters comprises selective activation of a subset of electrodes from the plurality of electrodes.

Example 24. The endovascular device of any of examples 1-23, wherein the plurality of electrodes comprises a coating configured to decrease an impedance of the plurality of electrodes.

Example 25. The endovascular device of any of examples 1-24, wherein the plurality of electrodes comprises a surface texture configured to decrease an impedance of the plurality of electrodes.

Example 26. A method comprising: detecting, via processing circuitry configured to control therapy delivery via a plurality of electrodes disposed along an elongated body, a level of endothelization proximate the plurality of electrodes based on a sensed parameter; and selecting, via the processing circuitry, one or more electrical stimulation parameter values based on detecting the level of endothelization proximate the plurality of electrodes, wherein the elongated body is configured to be introduced in a blood vessel of a patient, wherein the plurality of electrodes comprises a first group of electrodes and a second group of electrodes, wherein a first conductor is electrically coupled to each electrode of the first group of electrodes and a second conductor is electrically coupled to each electrode of the second group of electrodes, wherein each electrode of the first group of electrodes faces a first direction and each electrode of the second group of electrodes faces a second direction different from the first direction, wherein each electrode of the plurality of electrodes is a segmented electrode or a partial ring electrode, and wherein the plurality of electrodes is configured to deliver electrical stimulation to tissue of a brain of the patient or sense a patient parameter from a location within the blood vessel.

Example 27. The method of example 26, wherein the plurality of electrodes further comprises a third group of electrodes, wherein a third conductor is electrically coupled to each electrode of the third group of electrodes, and wherein each electrode of the third group of electrodes faces a third direction different from the first direction and the second direction.

Example 28. The method of example 27, wherein the first group of electrodes, the second group of electrodes, and the third group of electrodes are independently activatable.

Example 29. The method of any of examples 27-28, wherein each electrode of the first group of electrodes is circumferentially aligned along the elongated body, wherein each electrode of the second group of electrodes is circumferentially aligned along the elongated body, and wherein each electrode of the third group of electrodes is circumferentially aligned along the elongated body.

Example 30. The method of any of examples 27-29, wherein the first group of electrodes is separated from the second group of electrodes and the third group of electrodes by 120 degrees around the elongated body.

Example 31. The method of any of examples 27-30, wherein the first group of electrodes comprises three or more electrodes, the second group of electrodes comprises three or more electrodes, and the third group of electrodes comprises three or more electrodes.

Example 32. The method of any of examples 27-31, wherein a first electrode of the first group of electrodes, a first electrode of the second group of electrodes, and a first electrode of the third group of electrodes are disposed on the elongated body at a first distance from a distal-most end of the elongated body, wherein a second electrode of the first group of electrodes, a second electrode of the second group of electrodes, and a second electrode of the third group of electrodes are disposed on the elongated body at a second distance from the distal-most end of the elongated body, and wherein a third electrode of the first group of electrodes, a third electrode of the second group of electrodes, and a third electrode of the third group of electrodes are disposed on the elongated body at a third distance from the distal-most end of the elongated body.

Example 33. The method of any of examples 26-32, wherein the sensed parameter is an impedance.

Example 34. The method of any of examples 26-33, wherein one or more electrical stimulation parameters comprises one or more of a frequency, a power, or an amplitude of electrical stimulation.

Example 35. The method of any of examples 26-34, wherein one or more electrical stimulation parameters comprises selective activation of a subset of electrodes from the plurality of electrodes.

Various examples of the disclosure have been described. Any combination of the described systems, operations, or functions is contemplated. These and other examples are within the scope of the following claims.

What is claimed is:

1. An endovascular device comprising:
an elongated body configured to be introduced in a blood vessel of a patient;
a plurality of electrodes disposed along the elongated body, the plurality of electrodes comprising a first group of electrodes and a second group of electrodes; and
a plurality of conductors comprising a first conductor electrically coupled to each electrode of the first group of electrodes and a second conductor electrically coupled to each electrode of the second group of electrodes,
wherein each electrode of the first group of electrodes faces a first direction and each electrode of the second group of electrodes faces a second direction different from the first direction,
wherein each electrode of the plurality of electrodes is a segmented electrode or a partial ring electrode, and
wherein the plurality of electrodes is configured to deliver electrical stimulation to tissue of a brain of the patient or sense a patient parameter from a location within the blood vessel.

2. The endovascular device of claim 1,
wherein the plurality of electrodes further comprises a third group of electrodes,
wherein the plurality of conductors further comprises a third conductor electrically coupled to each electrode of the third group of electrodes, and
wherein each electrode of the third group of electrodes faces a third direction different from the first direction and the second direction.

3. The endovascular device of claim 2, wherein the first group of electrodes, the second group of electrodes, and the third group of electrodes are independently activatable.

4. The endovascular device of claim 2, wherein each electrode of the first group of electrodes is circumferentially aligned along the elongated body, wherein each electrode of the second group of electrodes is circumferentially aligned along the elongated body, and wherein each electrode of the third group of electrodes is circumferentially aligned along the elongated body.

5. The endovascular device of claim 2, wherein the first group of electrodes comprises three or more electrodes, the second group of electrodes comprises three or more electrodes, and the third group of electrodes comprises three or more electrodes.

6. The endovascular device of claim 2,
wherein a first electrode of the first group of electrodes, a first electrode of the second group of electrodes, and a first electrode of the third group of electrodes are disposed on the elongated body at a first distance from a distal-most end of the elongated body,
wherein a second electrode of the first group of electrodes, a second electrode of the second group of electrodes, and a second electrode of the third group of electrodes are disposed on the elongated body at a second distance from the distal-most end of the elongated body, and
wherein a third electrode of the first group of electrodes, a third electrode of the second group of electrodes, and a third electrode of the third group of electrodes are disposed on the elongated body at a third distance from the distal-most end of the elongated body.

7. The endovascular device of claim 1, further comprising a medical lead comprising the elongated body.

8. The endovascular device of claim 1, wherein the elongated body comprises an electrically insulative material, wherein the at least a portion of the electrically insulative material is removed to expose an electrically conductive material to define the plurality of electrodes.

9. The endovascular device of claim 1, wherein the endovascular device is configured to be operated in a trial mode for a trial period to determine an efficacy of the electrical stimulation or sensing.

10. The endovascular device of claim 1, further comprising one or more surface textures or coatings configured to promote endothelization within the blood vessel.

11. The endovascular device of claim 1, further comprising processing circuitry configured to control therapy delivery via the plurality of electrodes, wherein the processing circuitry is configured to detect a level of endothelization proximate the plurality of electrodes based on a sensed parameter.

12. The endovascular device of claim 11, wherein the sensed parameter is an impedance.

13. The endovascular device of claim 11, wherein the processing circuitry is configured to select one or more electrical stimulation parameters values based on detecting the level of endothelization proximate the plurality of electrodes.

14. The endovascular device of claim 13, wherein one or more electrical stimulation parameters comprises one or more of a frequency, a power, or an amplitude of electrical stimulation.

15. An endovascular device comprising:
an elongated body comprising a distal portion transformable between a relatively low-profile delivery configuration and a deployed configuration, wherein the distal portion is configured to be introduced in a blood vessel of a patient;
a plurality of electrodes disposed along the elongated body, the plurality of electrodes comprising a first group of electrodes and a second group of electrodes; and
a plurality of conductors comprising a first conductor electrically coupled to each electrode of the first group of electrodes and a second conductor electrically coupled to each electrode of the second group of electrodes,
wherein when the distal portion of the elongated body is in the deployed configuration, each electrode of the first group of electrodes faces a first direction and each electrode of the second group of electrodes faces a second direction different from the first direction, and
wherein the plurality of electrodes is configured to deliver electrical stimulation to tissue of a brain of the patient or sense a patient parameter from a location within the blood vessel.

16. The endovascular device of claim 15,
wherein the plurality of electrodes further comprises a third group of electrodes,
wherein the plurality of conductors further comprises a third conductor electrically coupled to each electrode of the third group of electrodes, and
wherein when the distal portion of the elongated body is in the deployed configuration, each electrode of the third group of electrodes faces a third direction different from the first direction and the second direction.

17. The endovascular device of claim 15, wherein in the relatively low-profile delivery configuration, each electrode of the plurality of electrodes is aligned along the elongated body.

18. The endovascular device of claim 15, wherein the endovascular device is configured to be operated in a trial mode for a trial period to determine an efficacy of the electrical stimulation or sensing.

19. The endovascular device of any of claim 15, further comprising processing circuitry configured to control therapy delivery via the plurality of electrodes, wherein the processing circuitry is configured to detect a level of endothelization proximate the plurality of electrodes based on a sensed parameter.

20. An endovascular device comprising:
a frame configured to expand from a relatively low-profile delivery configuration to a deployed configuration in a blood vessel of a patient;
a plurality of electrodes disposed along the frame, the plurality of electrodes comprising a first group of electrodes, a second group of electrodes, and a third group of electrodes; and
a plurality of conductors comprising a first conductor electrically coupled to each electrode of the first group of electrodes, a second conductor electrically coupled to each electrode of the second group of electrodes, and a third conductor electrically coupled to each electrode of the third group of electrodes,
wherein the first conductor, the second conductor, and the third conductor form the frame,
wherein each electrode of the first group of electrodes faces a first direction, each electrode of the second group of electrodes faces a second direction different from the first direction, and each electrode of the third group of electrodes faces a third direction different from the first direction and the second direction, and
wherein the plurality of electrodes is configured to deliver electrical stimulation to tissue of a brain of the patient or sense a patient parameter from a location within the blood vessel.

21. The endovascular device of claim 20, wherein the frame comprises a shape memory material, and wherein the frame is expandable.

22. The endovascular device of claim 20, wherein the endovascular device is configured to be operated in a trial mode for a trial period to determine an efficacy of the electrical stimulation or sensing.

23. The endovascular device of claim 20, further comprising processing circuitry configured to control therapy delivery via the plurality of electrodes, wherein the processing circuitry is configured to detect a level of endothelization proximate the plurality of electrodes based on a sensed parameter and select one or more electrical stimulation parameters values based on detecting the level of endothelization.

24. A method comprising:
detecting, via processing circuitry configured to control therapy delivery via a plurality of electrodes disposed along an elongated body, a level of endothelization proximate the plurality of electrodes based on a sensed parameter; and
selecting, via the processing circuitry, one or more electrical stimulation parameter values based on detecting the level of endothelization proximate the plurality of electrodes,
wherein the elongated body is configured to be introduced in a blood vessel of a patient,
wherein the plurality of electrodes comprises a first group of electrodes and a second group of electrodes,
wherein a first conductor is electrically coupled to each electrode of the first group of electrodes and a second conductor is electrically coupled to each electrode of the second group of electrodes,
wherein each electrode of the first group of electrodes faces a first direction and each electrode of the second group of electrodes faces a second direction different from the first direction,
wherein each electrode of the plurality of electrodes is a segmented electrode or a partial ring electrode, and
wherein the plurality of electrodes is configured to deliver electrical stimulation to tissue of a brain of the patient or sense a patient parameter from a location within the blood vessel.

25. The method of claim 24,
wherein the plurality of electrodes further comprises a third group of electrodes,
wherein a third conductor is electrically coupled to each electrode of the third group of electrodes, and
wherein each electrode of the third group of electrodes faces a third direction different from the first direction and the second direction.

26. The method of claim 24, further comprising selectively delivering electrical stimulation to tissue of the brain of the patient or selectively sensing the patient parameter via the first group electrodes and not delivering electrical stimulation to tissue of the brain of the patient or not sensing the patient parameter via the second group of electrodes or the third group of electrodes.

27. The method of claim 24, wherein each electrode of the first group of electrodes is circumferentially aligned along the elongated body, wherein each electrode of the second group of electrodes is circumferentially aligned along the elongated body, and wherein each electrode of the third group of electrodes is circumferentially aligned along the elongated body.
